(19) United States
(12) Patent Application Publication
(10) Pub. No.: US 2006/0247256 A1
(43) Pub. Date: Nov. 2, 2006

(54) Phenylpiperazine derivatives with a combination of partial dopamine-D2 receptor agonism and serotonin reuptake inhibition

(75) Inventors: Roelof W. Feenstra, Weesp (NL); Axel Stoit, Weesp (NL); Jan-Willem Terpstra, Weesp (NL); Maria L. Pras-Raves, Weesp (NL); Andrew C. McCreary, Weep (NL); Bernard J. Van Vliet, Weep (NL); Mayke B. Hesselink, Weep (NL); Cornelis G. Kruse, Weep (NL); Gustaaf J.M. Van Scharrenburg, Weep (NL)

Correspondence Address: FINNEGAN, HENDERSON, FARABOW, GARRETT & DUNNER;LLP, 901 NEW YORK AVENUE, NW, WASHINGTON, DC, 20001-4413, US

(73) Assignee: SOLVAY PHARMACEUTICALS B.V.

(21) Appl. No.: 11/294,615

(22) Filed: Dec. 6, 2005

Related U.S. Application Data
(60) Provisional application No. 60/634,074, filed on Dec. 8, 2004.

(51) Int. Cl. A61K 31/496; C07D 413/14

(52) U.S. Cl. 514/254.02; 544/368

(57) ABSTRACT

The invention relates to a group of novel phenylpiperazine derivatives with a dual mode of action: serotonin reuptake inhibition and partial agonism on dopamine-D₂ receptors. The invention also relates to the use of a compound disclosed herein for the manufacture of a medicament giving a beneficial effect.
The compounds have the general formula (1): 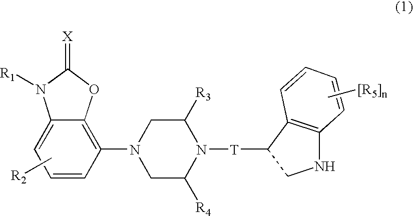 wherein the symbols have the meanings given in the specification.

The present invention relates to a group of novel phenylpiperazine derivatives with a dual mode of action: serotonin reuptake inhibition and partial agonism on dopamine-D₂ receptors. The invention also relates to the use of a compound disclosed herein for the manufacture of a medicament giving a beneficial effect. A beneficial effect is disclosed herein or apparent to a person skilled in the art from the specification and general knowledge in the art. The invention also relates to the use of a compound of the invention for the manufacture of a medicament for treating or preventing a disease or condition. More particularly, the invention relates to a new use for the treatment of a disease or condition disclosed herein or apparent to a person skilled in the art from the specification and general knowledge in the art. In embodiments of the invention specific compounds disclosed herein are used for the manufacture of a medicament useful in the treatment of disorders in which dopamine-D₂ receptors and serotonin reuptake sites are involved, or that can be treated via manipulation of those targets.

Compounds with a dual action as dopamine-D₂ antagonists and serotonin reuptake inhibitors are known from WO 00/023441, WO 00/069424 and WO 01/014330. This combination of activities is useful for the treatment of schizophrenia and other psychotic disorders: it enables a more complete treatment of all disease symptoms (e.g. positive symptoms and negative symptoms).

The goal of the present invention was to provide further compounds with a dual action as partial dopamine-D₂ antagonists and serotonin reuptake inhibitors.

The invention relates to a group of novel compounds of the formula (1): 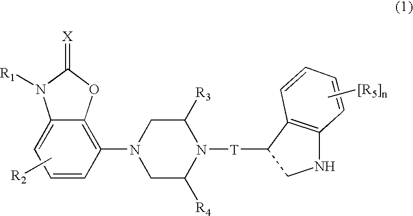
wherein:
X═S or O,
R₁ is H, (C₁-C₆)alkyl, CF₃, CH₂CF₃, OH or O—(C₁-C₆)alkyl
R₂ is H, (C₁-C₆)alkyl, halogen or cyano
R₃ is H or (C₁-C₆)alkyl
R₄ is H, (C₁-C₆)alkyl, optionally substituted with a halogen atom
   T is a saturated or unsaturated carbon chain of 2-7 atoms, wherein one carbon atom may be replaced with a nitrogen atom, optionally substituted with an (C₁-C₃)alkyl, CF₃ or CH₂CF₃ group, an oxygen atom or a sulphur atom, which chain is optionally substituted with one or more substituents selected from the group consisting of (C₁-C₃)alkyl, (C₁-C₃)alkoxy, halogen, cyano, trifluoromethyl, OCF₃, SCF₃, OCHF₂ and nitro,
   the dotted line is either a single or a double bond,
   R₅ is a substituent selected from the group consisting of (C₁-C₃)alkyl, (C₁-C₃)alkoxy, halogen, cyano, trifluoromethyl, OCF₃, SCF₃, OCHF₂ and nitro,
   n has the value 0-4,
   and tautomers, stereoisomers and N-oxides thereof, as well as pharmacologically acceptable salts, hydrates and solvates of said compounds of formula (1) and its tautomers, stereoisomers and N-oxides,
   with the proviso that when X═O, R₁, R₃ and R₄ are hydrogen, R₂ is hydrogen or halogen, and the group attached to T is an indolyl group, said indolyl group is substituted with one or more substituents selected from the group consisting of trifluoromethyl, OCF₃, SCF₃, OCHF₂ or nitro,

In the description of the substituents the abbreviation ‘alkyl(C₁₋₃)’ means ‘methyl, ethyl, n-propyl or isopropyl’.

Prodrugs of the compounds mentioned above are in the scope of the present invention. Prodrugs are therapeutic agents which are inactive per se but are transformed into one or more active metabolites. Prodrugs are bioreversible derivatives of drug molecules used to overcome some barriers to the utility of the parent drug molecule. These barriers include, but are not limited to, solubility, permeability, stability, presystemic metabolism and targeting limitations (Medicinal Chemistry: Principles and Practice, 1994, Ed.: F. D. King, p. 215; J. Stella, “Prodrugs as therapeutics”, Expert Opin. Ther. Patents, 14(3), 277-280, 2004; P. Ettmayer et al., “Lessons learned from marketed and investigational prodrugs”, J. Med. Chem., 47, 2393-2404, 2004). Pro-drugs, i.e. compounds which when administered to humans by any known route, are metabolised to compounds having formula (1), belong to the invention. In particular this relates to compounds with primary or secondary amino or hydroxy groups. Such compounds can be reacted with organic acids to yield compounds having formula (1) wherein an additional group is present which is easily removed after administration, for instance, but not limited to amidine, enamine, a. Mannich base, a hydroxyl-methylene derivative, an O-(acyloxy-methylene carbamate) derivative, carbamate, ester, amide or enaminone.

N-oxides of the compounds mentioned above are in the scope of the present invention. Tertiary amines may or may not give rise to N-oxide metabolites. The extend to what N-oxidation takes place varies from trace amounts to a near quantitative conversion. N-oxides may be more active than their corresponding tertiary amines or less active.

Whilst N-oxides are easily reduced to their corresponding tertiary amines by chemical means, in the human body this happens to varying degrees. Some N-oxides undergo nearly quantitative reductive conversion to the corresponding tertiary amines, in other cases the conversion is a mere trace reaction or even completely absent. (M. H. Bickel: “The pharmacology and Biochemistry of N-oxides”, Pharmacological Reviews, 21(4), 325-355, 1969).

It has been found that the compounds according to the invention show high affinity for both the dopamine D₂ receptor and the serotonin reuptake site. The compounds show activity at dopamine D₂ receptors with varying degree of agonism. All of the compounds show activity as inhibitors of serotonin reuptake, as they potentiate 5-HTP induced behaviour in mice (B. L. Jacobs., ‘An animal behaviour model for studying central serotonergic synapses’, Life Sci., 1976, 19(6) 777-785).

In contrast to the use of full dopamine-D₂ receptor agonists or antagonists, the use of partial dopamine-D₂ receptor agonists offers a dynamic medication that self-adjusts on a moment-to-moment basis to the endogenous state of the patient. Thus, it provides the desired flexible modulation of the dopamine system and avoidance of the many adverse effects caused either by treatment using full dopamine-D₂ receptor agonists like bromocriptine (hallucinations, nausea, vomiting, dyskinesia, orthostatic hypotension, somnolescence) or full dopamine-D₂ receptor antagonists like haloperidol (emotional blunting, dysphoria, tardive dyskinesia). Because of these many adverse effects, full agonists and antagonists have found only very limited use in the therapy of depressive and anxiety disorders. Partial dopamine-D₂ receptor agonists not only show a flexible modulation and a favourable side-effect profile, they also have a pronounced anxiolytic profile in relevant animal models (Drugs of the Future 2001, 26(2): 128-132).

Partial dopamine-D₂ receptor agonists, according to the present invention, are compounds that—when tested in a concentration response range—achieve activation in the functional cAMP cell based assay (as described below). Partial dopamine-D₂ receptor agonists will act as an agonist in cases when the endogenous synaptic tone of dopamine is low, or in the the presence of a full dopamine-D₂ receptor antagonist, and will act as an antagonist in cases when the endogenous synaptic tone of dopamine is high, or in the presence of a full dopamine D₂ receptor agonist. Like full agonists, partial dopamine-D₂ receptor agonists in general are active in sensitized systems. They induce contralateral turning in rats with unilateral 6-hydroxy-dopamine (6-OHDA) lesions in the substantia nigra pars compacta. In MPTP-treated common marmosets they produce potent and long-lasting reversal of motor symptoms (Drugs of the Future 2001, 26(2): 128-132). In contrast to full agonists, however, partial dopamine-D₂ agonists are substantially less active in non-sensitized systems: they hardly reverse reserpine induced hypolocomotion in rats.

For the treatment of CNS disorders involving an overactive dopaminergic system a pharmaceutical preparation combining partial dopamine-D₂ receptor agonistic activity having low intrinsic functional activity with serotonin reuptake inhibitory activity is recommended. In case of a disorder involving dopamine insufficiency a pharmaceutical preparation combining partial dopamine-D₂ receptor agonistic activity with high intrinsic functional activity and serotonin reuptake activity according to the invention has considerable advantages.

Disorders characterized by dynamic fluctuations in dopamine neurotransmission like bipolar depression and addiction will profit in particular from the flexible adjustment of the dopamine system by the partial dopamine-D₂ receptor agonists in the pharmaceutical preparation. Combining this “dopaminergic neurotransmission stabilizing” activity with serotonin reuptake inhibitory activity will enhance antidepressive and anxiolytic efficacy. The compounds can be used for the treatment of affections or diseases of the central nervous system caused by disturbances in the dopaminergic and serotonergic systems, for example: aggression, anxiety disorders, autism, vertigo, depression, disturbances of cognition or memory, Parkinson's disease, and in particular schizophrenia and other psychotic disorders.

Pharmaceutically acceptable salts may be obtained using standard procedures well known in the art, for example by mixing a compound of the present invention with a suitable acid, for instance an inorganic acid such as hydrochloric acid, or with an organic acid.

Pharmaceutical Preparations

The compounds of the invention can be brought into forms suitable for administration by means of usual processes using auxiliary substances such as liquid or solid carrier material. The pharmaceutical compositions of the invention may be administered enterally, orally, parenterally (intramuscularly or intravenously), rectally or locally (topically). They can be administered in the form of solutions, powders, tablets, capsules (including microcapsules), ointments (creams or gel) or suppositories. Suitable excipients for such formulations are the pharmaceutically customary liquid or solid fillers and extenders, solvents, emulsifiers, lubricants, flavorings, colorings and/or buffer substances. Frequently used auxiliary substances which may be mentioned are magnesium carbonate, titanium dioxide, lactose, mannitol and other sugars, talc, lactoprotein, gelatin, starch, cellulose and its derivatives, animal and vegetable oils such as fish liver oil, sunflower, groundnut or sesame oil, polyethylene glycol and solvents such as, for example, sterile water and mono- or polyhydric alcohols such as glycerol.

Compounds of the present invention are generally administered as pharmaceutical compositions which are important and novel embodiments of the invention because of the presence of the compounds, more particularly specific compounds disclosed herein. Types of pharmaceutical compositions that may be used include but are not limited to tablets, chewable tablets, capsules, solutions, parenteral solutions, suppositories, suspensions, and other types disclosed herein or apparent to a person skilled in the art from the specification and general knowledge in the art. In embodiments of the invention, a pharmaceutical pack or kit is provided comprising one or more containers filled with one or more of the ingredients of a pharmaceutical composition of the invention. Associated with such container(s) can be various written materials such as instructions for use, or a notice in the form prescribed by a governmental agency regulating the manufacture, use or sale of pharmaceuticals products, which notice reflects approval by the agency of manufacture, use, or sale for human or veterinary administration.

Pharmacological Methods

In Vitro Affinity for Dopamine-D₂ Receptors

Affinity of the compounds for dopamine-D₂ receptors was determined using the receptor binding assay described by I. Creese, R. Schneider and S. H. Snyder: “[³H]-Spiroperidol labels dopamine receptors in rat pituitary and brain”, Eur. J. Pharmacol., 46, 377-381, 1977.

In Vitro Affinity for Serotonin Reuptake Sites

Affinity of the compounds for serotonin reuptake sites was determined using the receptor binding assay described by E. Habert et al.,: “Characterisation of [³H]-paroxetine binding to rat cortical membranes”, Eur. J. Pharmacol., 118, 107-114, 1985.

Inhibition of Forskolin-Induced [³H]-cAMP Accumulation

The in vitro functional activity at dopamine-D₂ receptors, including the intrinsic activity (O) of the compounds of the invention was measured by their ability to inhibit forskolin-induced [³H]-cAMP accumulation.

Human dopamine D_{2,L} receptors were cloned in fibroblast cell line CHO-K1 cells and obtained from Dr. Grandy, Vollum Institute, Portland, Oreg., USA. CHO cells were grown in a Dulbecco's modified Eagle's medium (DMEM) culture medium, supplemented with 10% heat-inactivated fetal calf serum, 2 mM glutamine, 1 mM pyruvate, 5000 units/ml penicillin, 5000 μg/ml streptomycin and 200 μg/ml G-418 at 37° C. in 93% air/7% CO₂. For incubation with test compounds, confluent cultures grown in 24 wells plates were used. Each condition or substance was routinely tested in quadruplicate. Cells were loaded with 1 μCi [³H]-adenine in 0.5 ml medium/well. After 2 hours, cultures were washed with 0.5 ml PBS containing 1 mM of the phosphodiesterase inhibitor isobutylmethylxanthine (IBMX) and incubated for 20 min with 0.5 ml PBS containing 1 mM IBMX and forskolin with or without test compound. After aspiration the reaction was stopped with 1 ml trichloroacetic acid 5% (w/v). The [³H]-ATP and [³H]-cAMP formed in the cellular extract were assayed as described by Solomon Y, Landos C, Rodbell M, 1974, A highly selective adenylyl cyclase assay, Anal Biochem 58:541-548 and Weiss S, Sebben M, Bockaert J J, 1985, Corticotropin-peptide regulation of intracellular cyclic AMP production in cortical neurons in primary culture, J Neurochem 45:869-874. 0.8 ml Extract was passed over Dowex (50WX-4 200-400 mesh) and aluminumoxide columns, eluted with water and 0.1M imidazole (pH=7.5). Eluates were mixed with 7 ml Insta-gel and radioactivity was counted with a liquid scintillation counter. The conversion of [³H]-ATP into [³H]-cAMP was expressed as the ratio in percentage radioactivity in the cAMP fraction as compared to combined radioactivity in both cAMP and ATP fractions, and basal activity was subtracted to correct for spontaneous activity.

Test compounds were obtained as 10 mM stock solutions in 100% DMSO, and diluted in PBS/IBMX to final concentrations. Typically, compounds were used in concentrations that ranged from 10⁻¹⁰M to 10⁻⁵M. From quadruplicate data counts, the mean was taken as an estimate for drug-induced, receptor-mediated effects at specified second messenger accumulation, expressed as percentage of control values (forskolin-stimulated cAMP accumulation, subtracted by basal activity). By using the non-linear curve-fitting program INPLOT or the Excel-add-in XL-Fit, mean values were plotted against drug concentration (in molar) and a sigmoid curve (four-parameter logistic curve) was constructed. The maximal forskolin-induced stimulated conversion is taken as maximum value and the maximal inhibition (usually at drug concentrations 10⁻⁶ M or 10⁻⁵ M) as minimum and these values were fixed during the fitting process. Thus, concentrations of the compound, causing 50% of the maximally obtained inhibition of forskolin-induced cAMP accumulation (EC₅₀), are averaged over several experiments and presented as mean pEC₅₀±SEM. Antagonist potency is assessed by co-incubating cells with a fixed agonist concentration and specified antagonist concentrations. Curve fitting procedures are identical to those used for estimating EC₅₀ values. Thus IC₅₀ values, i.e. the concentration that is able to achieve 50% of maximal antagonism that can be achieved by this compound. IC₅₀ values are corrected using a Cheng-Prussoff equation, correcting it for agonist concentration and EC₅₀ values that is obtained in the same experiment. Thus, K_{b}=IC₅₀/(1+[agonist]/EC₅₀, agonist). The corresponding pA₂ value is −log (K_{b}). Concentration-response curve fitting allows estimation of pEC₅₀ values and of maximal achievable effect (intrinsic activity or efficacy (ε). A full receptor agonist has ε=1, a full receptor antagonist has ε=0, and a partial receptor agonist has an intermediate intrinsic activity.

Dosages

The affinity of the compounds of the invention for dopamine-D₂ receptors and serotonine reuptake sites was determined as described above. From the binding affinity measured for a given compound of formula (1), one can estimate a theoretical lowest effective dose. At a concentration of the compound equal to twice the measured Kᵢ-value, 100% of the receptors likely will be occupied by the compound. Converting that concentration to mg of compound per kg of patient yields a theoretical lowest effective dose, assuming ideal bioavailability. Pharmacokinetic, pharmacodynamic, and other considerations may alter the dose actually administered to a higher or lower value. The dosage expediently administered is 0.001-1000 mg/kg, preferably 0.1-100 mg/kg of patient's bodyweight.

Treatment

The term ‘treatment’ as used herein refers to any treatment of a mammalian, preferably human condition or disease, and includes: (1) preventing the disease or condition from occurring in a subject which may be predisposed to the disease but has not yet been diagnosed as having it, (2) inhibiting the disease or condition, i.e., arresting its development, (3) relieving the disease or condition, i.e., causing regression of the condition, or (4) relieving the conditions caused by the disease, i.e., stopping the symptoms of the disease.

The preparation of the compounds having formula (I) will now be described in more detail in the following Examples.

### EXAMPLES

The H-atom of the N—H moiety of the phenylpiperazine part of the compounds of formula (1), the ‘amines’ I-H to X-H can be replaced by 0 in three different chemical ways, A, B and C, eventually leading to the compounds of the invention which are listed in table 1 (see below).

Method A:

The compounds were prepared via the synthesis depicted in scheme A1: an amine was reacted with Q-X (X=leaving group like e.g. Cl, Br, I) in e.g. acetonitrile or butyronitrile with Et(i-Pr)₂N acting as a base, in some cases KI (or NaI) was added. Et₃N can be used instead of Et(i-Pr)₂N.

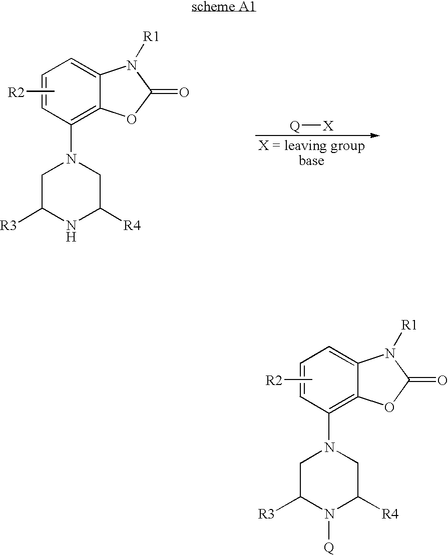

### Example 1

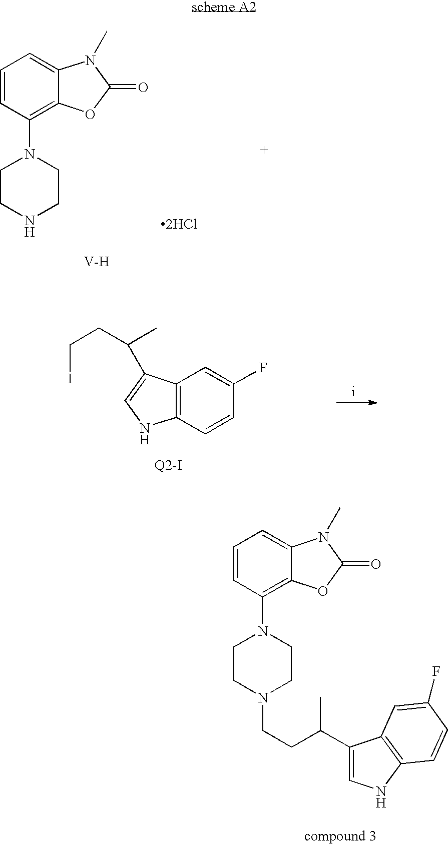 Scheme A2, Step i:

A mixture of 0.6 g (1.96 mmol) of the dihydrochloride of piperazine V-H.2HCl, 0.62 g (1.96 mmol) of the iodide O₂-I, 0.6 g (4 mmol) of NaI and 1.5 ml (8.6 mmol) of DIPEA in 100 ml of acetonitril was refluxed for 20 hours. After concentration in vacuo, the residu was taken up in CH₂Cl₂ and the latter fraction washed with water. The organic fraction was dried (Na₂SO₄). After removal of the drying agent by filtration and solvent by concentration in vacuo, the residu was subjected to flash column chromatography (SiO₂, eluent: CH₂Cl₂/MeOH/NH₄OH 960/37.5/2.5) yielding the pure free base 3. The latter was converted into its HCl salt (by treatment with 1 eq. of 1.0 N AcCl/MeOH), giving compound 3.HCl, melting point 100-140° C. (decomposition).

Method B:

The compounds listed in table 1 (see below) were prepared via the synthesis depicted in scheme B1: an amine was alkylated by means of a reductive alkylation. Q-OH was oxidized to the corresponding aldehyde Q′-CHO after which the reductive alkylation was performed. THF and DCE are suitable solvents for this type of reaction.

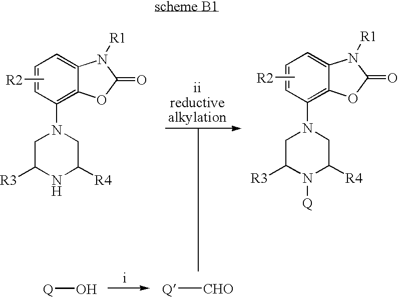

### Example 2

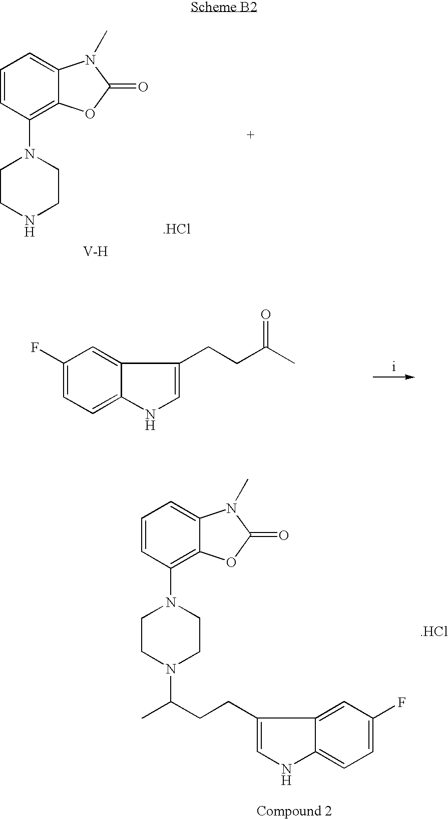 Scheme B2, Step i;

To a stirred suspension of V-H.HCl (0.68 g, 2.53 mmol) and ketone (0.47 g, 2.3 mmol) in 15 ml of THF stirred under a nitrogen atmosphere was added: triethylamine (0.27 g, 0.37 ml, 2.66 mmol), NaBH(OAc)₃ (0.76 g, 3.6 mmol) and ACOH (0.26 g, 0.26 ml, 4.6 mmol). The suspension was stirred at room temperature for 110 hours. The reaction mixture was poured into a 5% NaHCO₃-solution and the resulting mixture was extracted three times with EtOAc. The combined organic fractions were washed with brine and dried (Na₂SO₄). After removal of the drying agent by filtration and the solvent by concentration in vacuo, the residu was subjected to flash column chromatography (SiO₂, eluent DCM/MeOH 97/3) giving 0.33 g foam which was dissolved in EtOAc and treated with 0.85 ml 1.0 N HCl in EtOH to give 0.34 g of still impure 2.HCl. This was recrystallised from hot 30 ml Et₂O/EtOAc (2/1) to give 0.21 g of pure compound 2.HCl as a white solid. Melting point: 207-9° C.

Method C:

This method is dedicated to compound 17 only.

### Example 3

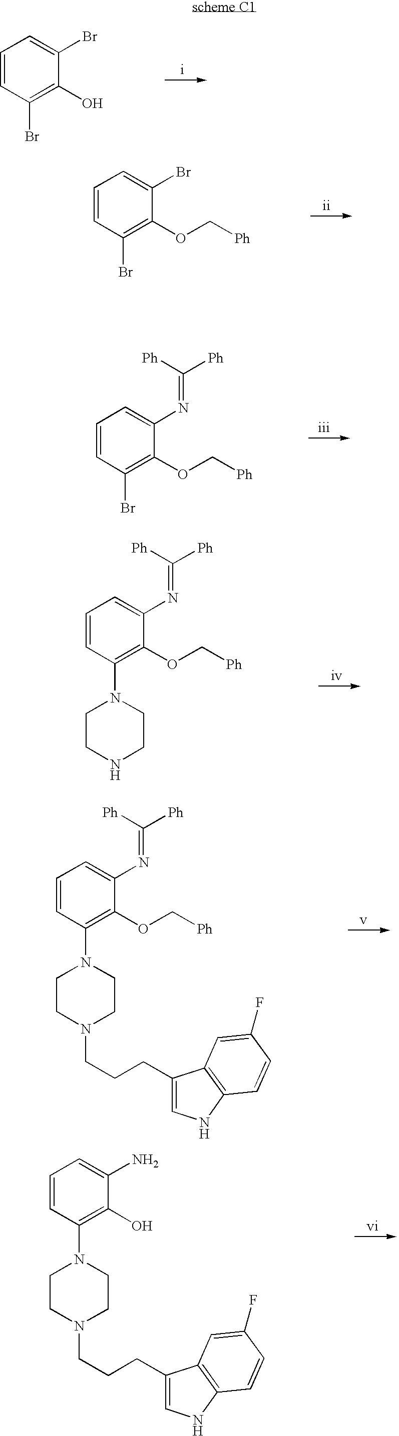 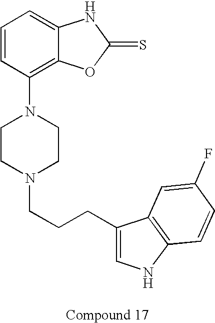 Scheme C1, Step i:

This step was done analogously to step i in scheme IV.

Scheme C1, Step ii:

This step was done analogously to step ii in scheme IV using benzophenonimine as the amine. After work up, the residu should be treated carefully; chromatographic purification was carried out by using Al₂O₃ (neutral, activity IV, Aldrich), eluent: DCM/petroleum ether ¼, yielding the protected aniline derivative in 76% yield as a yellow oil which solidifies upon standing.

Scheme C1, Step iii:

This step was done analogously to step ii in scheme IV using piperazine as the amine. After work up the residu was purified by flash column chromatography (Al₂O₃ (neutral, activity IV, Aldrich) eluent: DMA 0.125), eventually yielding a brown oil which was not pure. A second flash column chromatography (Al₂O₃ (neutral, activity IV, Aldrich) eluent: DMA 0.25→DMA 0.50 yielded a brown-yellowish oil in a yield of 65% containing the phenylpiperazine derivative.

Scheme C1, Step iv:

4.47 g (10 mmol) of the phenylpiperazine derivative (from step iii), 3.25 g of the iodide Q9-1 and 1.94 g (15 mmol) of DIPEA were taken up in 175 ml of acetonitrile and the mixture was refluxed for 18 hours. After cooling to room temperature, the reaction mixture was concentrated in vacuo, after which the residu was taken up in water and DCM. The water fraction was extracted with DCM. The collected organic fractions were washed with water and brine, then dried on Na₂SO₄. After removal of the drying agent by filtration and the solvent by evaporation, the resulting residu was purified by flash column chromatography (Al₂O₃ (neutral, activity IV, Aldrich) eluent: DMA 0.187), yielding a 5.0 g (80%) of a brown yellow foam containing the alkylated phenylpiperazine.

Scheme C1, Step v:

3.15 g (5.05 mmol) of the alkylated phenylpiperazine (from step iv) were dissolved in 100 ml of methanol, to the latter solution, 6.37 g (100 mmol) of ammonium formiate and a small amount of 10% Pd—C were added. The reaction mixture was refluxed for 20 hours, after cooling the mixture was filtered and the filtrate concentrated in vacuo. The residu was taken up in methanol and the latter solution passed through a SCX (ion-exchange) column (2×70 grams columns). Subsequent elution with 1M NH₃/MeOH released the desired product. Concentration of the product containing fractions yielded 0.82 g (44%) of a dark red glassy compound (containing the corresponding aminophenol) which was directly used in step vi.

Scheme C1, Step vi:

0.82 g (2.22 mmol) of the aminophenol (from step v), and 0.595 g (3.33 mmol) of thiocarbonyldiimidazol were dissolved in 25 ml of dry THF, after which the mixture was refluxed for 4 hours. After cooling down, the reaction mixture was concentrated in vacuo and the residu purified by flash column chromatography (SiO₂, eluent: DMA 0.50), yielding 1.27 g of a solid which was recrystalized from acetonitril giving 0.51 g of compound 17. Melting point: 238-240° C. (decomposition).

TABLE 1
examples of compounds of the invention.
Structures of the phenylpiperazine part of the compounds
of formula (1), herein termed ‘amines’, and groups ‘Q’ are
given below. In the column ‘method’, the general
method (A, B or C) is given, and in case of
method A, the next column gives the leaving group.

| comp. | amine | group Q | meth. | L-group | salt | melting r. ° C. |
|---|---|---|---|---|---|---|
| 1 | IV | 9 | A | I | HCl | 270-5 |
| 2 | V | 1 | B | | HCl | 207-209 |
| 3 | V | 2 | A | I | HCl | 100-140d |
| 4 | V | 3 | A | I | free base | 166-168 |
| 5 | V | 5 | A | Br | free base | 167-169 |
| 6 | V | 6 | A | I | free base | 159-162 |
| 7 | V | 7 | A | I | free base | 140-142 |
| 8 | V | 8 | A | I | HCl | >225 d |
| 9 | V | 9 | A | I | free base | 135-137 |
| 10 | V | 11 | A | I | free base | 151-152 |
| 11 | V | 12 | A | I | HCl | 254-256 |
| 12 | V | 13 | A | I | HCl | 213-215 |
| 13 | V | 14 | A | I | free base | 198-200 |
| 14 | VI | 8 | A | I | free base | 149-50 |
| 15 | VII | 9 | A | I | HCl | 246-9 |
| 16 | IX | 9 | A | I | HCl | 100-140 |
| 17 | X | 9 | C | | free base | 238-240d |

The phenylpiperazine parts of the compounds of formula (1) used in these methods are indicated as I-H to X-H, wherein the dot on the N-atom is the attachment point for the group Q:

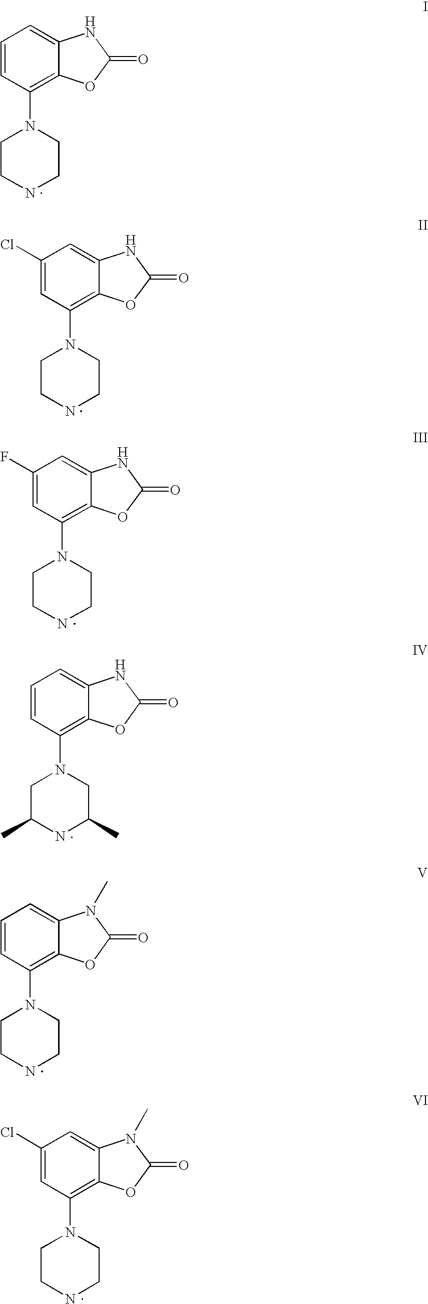

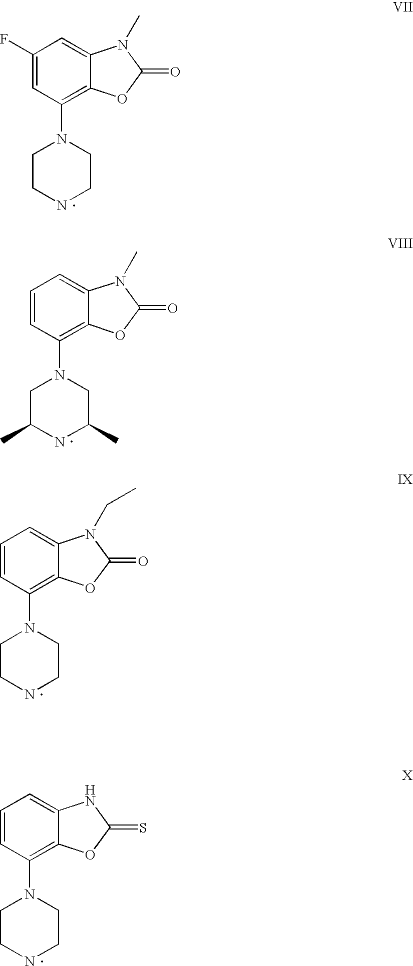

The synthesis of the piperazines I-H, III-H and V-H are described in WO97/36893.
Synthesis of Amine II-H:

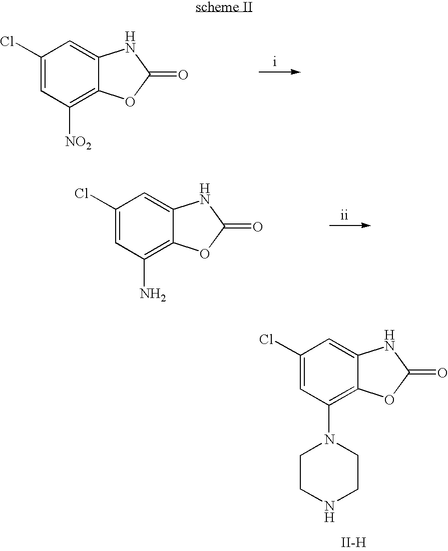

The synthesis of the starting material has been described (patent DE487014).

Scheme II, Step i:

30 g (0.14 mol) of the starting material was suspended in 600 ml of MeOH. Then a small amount of Raney nickel was added after which hydrogenation was started (atmospheric, room temperature). After 24 hours 7.2 liters (theoretical amount 9.4 liters) of hydrogen was absorbed. To the reaction mixture 150 ml of THF was added and another small amount of Raney nickel. After one hour the reaction mixture was filtered over hyflo, the residu washed with THF. The filtrate was concentrated in vacuo, yielding 25.2 g (98%) of the correspondig aniline.

Scheme II, Step ii:

24.2 g (131.2 mmol) of the aniline of the previous step and 25.8 g (144.3 mmol) of bis (2-chloroethyl)amine were suspended in 675 ml of chlorobenzene. While stirring, 25 ml of solvent were distilled off with the aid of a Dean-Stark apparatus. After removal of the Dean-Stark apparatus, the reaction was allowed to reflux for 48 hours. When the reaction mixture had come to room temperature, the mixture was decanted and the residu washed twice with Et₂O. Then 400 ml of MeOH were added after which the mixture was warmed until almost all of the residu was dissolved. Then 200 ml of silica were added after which the whole was concentrated in vacuo. Then the residu was put on top of a flash chromatography column using DMA 0.75 as the eluent. After removal of the solvent a residu was isolated which was suspended in about 100 ml of acetonitrile and stirred for 4 hours. Filtration and drying yielded 17 g of the desired piperazine II-H as a free base.
Synthesis of Amine IV-H:

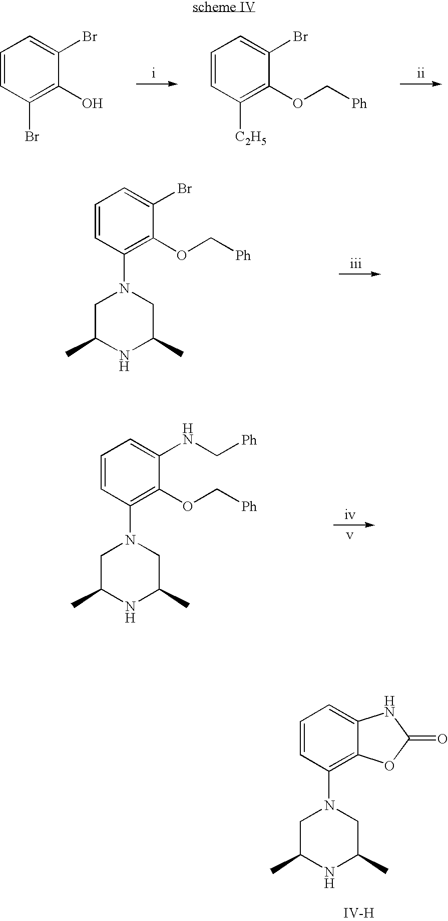

The toluene used in this experiment was degassed for three hours prior to usage. 1.48 g (1.61 mmol) of Pd₂(dba)₃ and 3.02 g (4.85 mmol) of BINAP were put into 400 ml of toluene after which the mixture was stirred and heated to 105° C. for 0.5 hours after which the mixture was allowed to room temperature. Subsequently were added to the reaction mixture: 27.

Scheme IV, Step i:

20.5 g (81.3 mmol) of dibromophenol and 20 g of potassium carbonate were suspended in 400 ml of aceton, after which 15.7 ml of benzylbromide were added. The reaction mixture was refluxed for 24 hours. After the mixture had reached room temperature, it was concentrated in vacuo. Subsequently water was added and CH₂Cl₂. The organic layer was filtered with a water repellant filter, the dry filtrate concentrated in vacuo after which it was dissolved again in 200 ml of acetonitrile. Subsequently, 15 ml of piperidine were added after which the temperature was raised to 60° C. for one hour. The reaction mixture was concentrated in vacuo and CH₂Cl₂ was added. The latter was washed with: 1N HCl (3×), water, 2N NaOH, and again water. The organic layer was filtered with a water repellant filter, the dry filtrate concentrated in vacuo yielding 27.6 g (99%) of the corresponding benzylated phenol.

Scheme IV, Step ii:

6 g (80.7 mmol) of the benzylated compound (step i) dissolved in 50 ml of toluene, 9.2 g (80.7 mmol) of the (α,α′)-dimethylpiperazine and 10.08 g (104.9 mmol) of sodium tert.butoxide. The resulting mixture was heated at 105° C. for 20 hours, after which it was allowed to reach room temperature. The mixture was diluted with CH₂Cl₂ after which it was filtered over hyflo and concentrated in vacuo. The residu was put on top of a flash chromatography column (SiO₂) using DMA 0.125. The combined product containing fractions yielded after concentration in vacuo 7.7 g (26%) of the almost pure phenylpiperazine.

Scheme IV, Step iii:

This step was done analogously to the procedure described in the previous step ii (scheme IV). In this case benzylamine was used in the Buchwald reaction. Yield: 88%.

Scheme IV, Step iv:

7 ml (98 mmol) of acetyl chloride was added dropwise to 70 ml of cooled absolute ethanol, stirring was continued for 15 minutes. The latter solution was added to a solution of 11.5 g (28.7 mmol) of the dibenzyl product of step iii in 250 ml of methanol. Subsequently 1.5 g of Pd/C (10%) was added, after which the reaction mixture was hydrogenated for 24 hours. The mixture was filtered over hyflo, the filtrate concentrated in vacuo. The residu containing the amino phenol HCl salt was directly used in step v.

Scheme IV, Step v:

The residu (28.7 mmol) obtained in step iv, 52 ml of DIPEA (298 mmol), and 20.9 g (129 mmol) of CDI were added to 750 ml of THF after which the mixture was refluxed for 20 hours under a nitrogen atmosphere. After cooling to room temperature, the mixture was concentrated in vacuo, to the residu CH₂Cl₂ and 5% NaHCO₃ were added, the whole being stirred for one hour. Extraction with CH₂Cl₂ (3×), the water fraction was concentrated and extracted again (CH₂Cl₂, 3×). The combined organic fractions were concentrated in vacuo, the residu contained a considerable amount of imidazol. The whole was solved in 120 ml of acetonitrile after which the solution was allowed to reach room temperature. The precipitate which formed was filtered yielding almost pure piperazine IV.
Synthesis of Amine V-H: 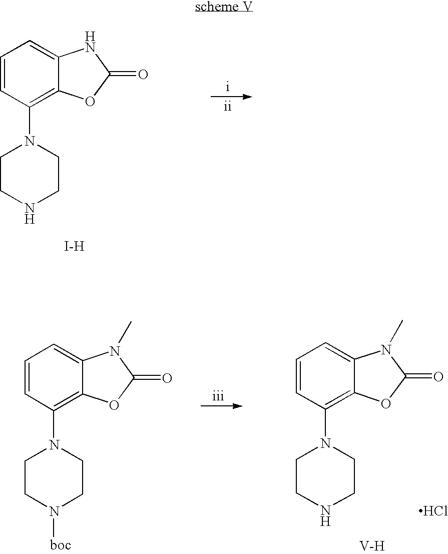
Scheme V, Steps i, ii and iii:

Synthesis of V-H has been described in WO97/36893. The steps i, ii and iii were done analogously to steps i, ii and iii in scheme VI.
Synthesis of Amine VI-H: 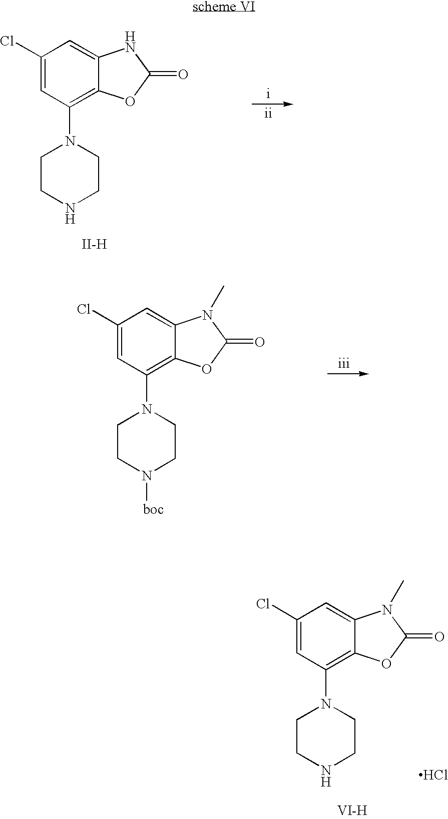
Scheme VI, Step i:

While stirring, 3.8 g (15 mmol) of piperazine II-H were suspended in 5.48 ml (31.5 mmol) of DIPEA and the mixture was brought to −40° C. A solution of 3.14 g (14.4 mmol, 0.96 eq) of Boc-anhydride in 30 ml of CH₂Cl₂ was added dropwise in 100 minutes. Stirring was continued at −40° C. (1 hour), then at −30° C. (2 hours), and the reaction mixture was allowed to come to room temperature (16 hours). Then water and some MeOH were added after which it was extracted with CH₂Cl₂. The combined organic fractions were filtered with a water repellant filter, the dry filtrate mixed with 50 ml of silica after which the whole was concentrated in vacuo. Then the residu was put on top of a dry chromatography column (SiO₂) using CH₂Cl₂/MeOH (98/2) as the eluent. The part of the column containing the product was cut out, and the product washed out of the column material with CH₂Cl₂/MeOH (98/2) yielding 3.55 g (67%) of the desired N-Boc II.

Scheme VI, Step ii:

4.5 g (12.7 mmol) N-Boc II together with 5.8 g (3.3 eq) of potassium carbonate were suspended in 100 ml of aceton. While stirring, the reaction mixture was cooled to −10° C. after which 0.87 ml (14 mmol, 1.1 eq) of methyl iodide was added dropwise. After 15 minutes, the reaction mixture was allowed to reach room temperature and stirring was continued for 14 hours. Subsequently, the reaction mixture was concentrated in vacuo, the residu mixed with water and CH₂Cl₂. The water layer was separated and extracted twice with CH₂Cl₂. The combined organic layers were filtered with a water repellant filter, the dry filtrate concentrated in vacuo yielding 4.5 g (98%) of the corresponding N′-methylated N-Boc II.

Scheme VI, Step iii:

While stirring at −10° C., 5 ml of acetyl chloride (70.4 mmol, 5.8 eq) was added dropwise to 65 ml of ethanol. The latter solution was added to 4.5 g (12.2 mmol) of the N′-methylated N-Boc II isolated in step ii. The resulting mixture was stirred for 3 hours at 55° C., then the reaction mixture was allowed to reach room temperature and stirring was continued for 14 hours. Subsequently, the mixture was concentrated in vacuo after which the residu was suspended in di-isopropyl ether and stirred for 2 hours. The precipitate was isolated by filtration yielding 3.6 g (97%) of piperazine VI-H.HCl.
Synthesis of Amine VII-H: 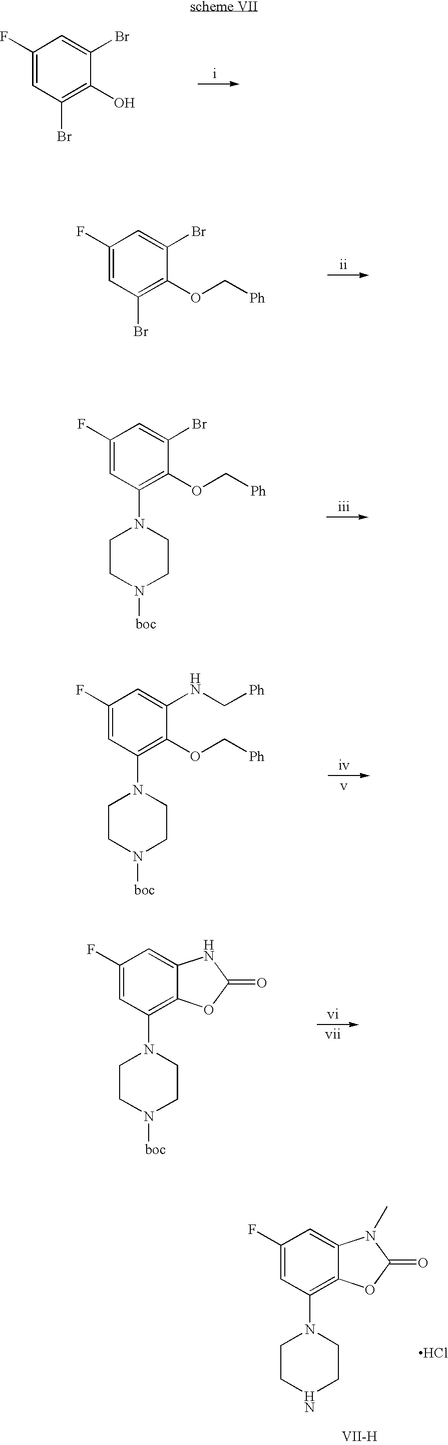
Scheme VII. Step i:

This step was done analogously to step i in scheme IV. After chromatograhic purification an oil containing the benzylated product, was isolated in 88% yield. The oil solidified upon standing.

Scheme VII, Step ii:

This step was done analogously to step ii in scheme IV. Boc-piperazine was used in this Buchwald reaction. Yield after chromatographic purification: 44% of a brown oil. PS Scheme VII, Step iii:

This step was done analogously to the procedure described in the previous step ii (scheme VII). In this case benzylamine was used in the Buchwald reaction. Yield after chromatographic purification: 73% of a brown oil.

Scheme VII, Step iv:

11.91 g (24.3 mmol) of the dibenzylated product isolated in previous step iii (scheme VII) was suspended in a mixture of 110 ml of ethanol, 72 ml of water and 11 ml of acetic acid. While stirring, 0.5 g of Pd(OH)₂/C was added and hydrogenation was started for 6 days. After one day and after 3 days an additional small amount of Pd(OH)₂/C was added. The reaction mixture was filtered over hyflo, the filtrate concentrated in vacuo. The residu was treated with toluene and concentrated in vacuo, this procedure was repeated, leaving a dark sirup 7.9 g (88%), containing the amino phenol.

Scheme VII, Step v:

This step (ring closure with CDI) was done analogously to step v in scheme IV. The crude product after work up was chromatographed (flash column, SiO₂, eluent DCM/MeOH 97/3) yielding 7.6 g of an impure brown foam. A second chromatography (flash column, SiO₂, eluent EtOAc/petroleum ether ½) yielded 3.3 g (42%) of pure brown foam, containing the N-Boc protected benzoxazolinone piperazine.

Scheme VII, Step vi:

This methylation step was done analogously to the procedure described in step ii (scheme VI). Yield: 98% of a brown foam of 97% purity.

Scheme VII, Step vii:

This deprotection step was done analogously to the procedure described in step iii (scheme VI). Yield: 94% of a light pink solid of 98% purity, containing the product VII-H.HCl.
Synthesis of Amine VIII-H: 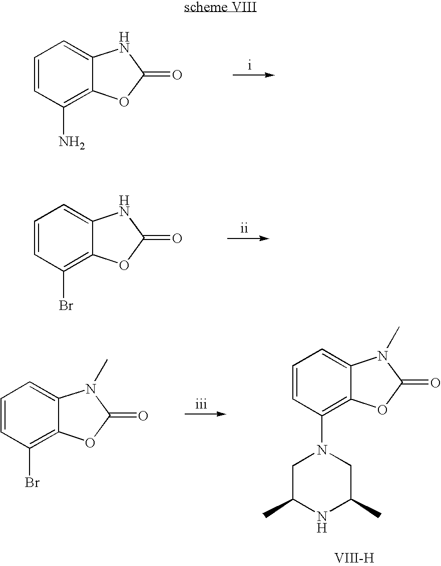
Scheme VIII, Step i:

The starting material synthesis has been described in EP0189612.

4.91 g (32.7 mmol) of the anilin was suspended in 75 ml of 48% of HBr/water, while it was cooled to −5° C. Subsequently 2.27 g (33 mmol) of sodium nitrite dissolved in 4 ml of water, were added dropwise during 15 minutes. Stirring was continued at 0° C. for 15 minutes.

Subsequently, the reaction mixture was added, in one time, to a 0° C. solution of 2.42 g (16.9 mmol) CuBr in 20 ml of 48% HBr/water. After 30 minutes the reaction mixture was heated to 85° C. for one hour, after which it was allowed to reach room temperature, stirring was continued for 14 hours. To the mixture diethyl ether and water were added, after shaking the organic layer was isolated which was washed with water. The organic layer, together with some silica, was concentrated in vacuo, and the residu was put on top of a flash chromatography column (SiO₂) using Et₂O/petroleum ether (1/1), and later on pure Et₂O as the eluent. The combined product containing fractions yielded after concentration in vacuo 3.3 g (47%) of the desired corresponding bromo product.

Scheme VIII, Step ii:

This step was carried out identical to step ii in scheme VI. Yield: 92% of the corresponding methylated bromo compound.

Scheme VIII, Step iii:

In the following order 6.82 g (29.9 mmol) of the methylated bromo compound, 4.03 g (35.9 mmol) of the dimethyl piperazine, 13.6 g (41.9 mmol) of Cs₂CO₃, 1.42 g (2.99 mmol) of X-Phos (see Huang et al., J. Am. Chem. Soc., 125(2003)6653) and 0.55 g (0.6 mmol) of Pd₂(dba)₃ were added to 225 ml of toluene which was degassed for 4 hours prior to usage. While stirring and under a nitrogen atmosphere the temperature was raised to 100° C. for 20 hours, after which it was allowed to reach room temperature. The mixture was diluted with CH₂Cl₂ after which it was filtered and concentrated in vacuo. The residu was put on top of a flash chromatography column (SiO₂) using DMA 0.25. The combined product containing fractions yielded after concentration in vacuo 0.73 g (9%) of the desired pure piperazine VIII-H.
Synthesis of Amine IX-H: 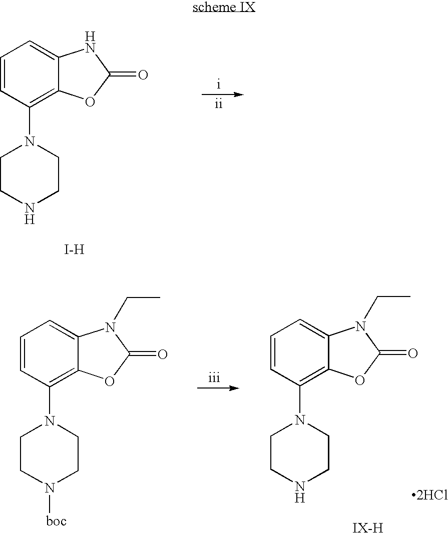
Scheme IX, Steps i, ii and iii:

Synthesis of I-H has been described in WO97/36893. The steps i, ii and iii were done analogously to steps iii and iii in scheme VI.

Synthesis of Amine X-H:

Piperazine X-H was not prepared, but built up during the synthesis of the complete compound 17, which is depicted in scheme C1.

Below, the different structures of Q1 to Q14 are given.

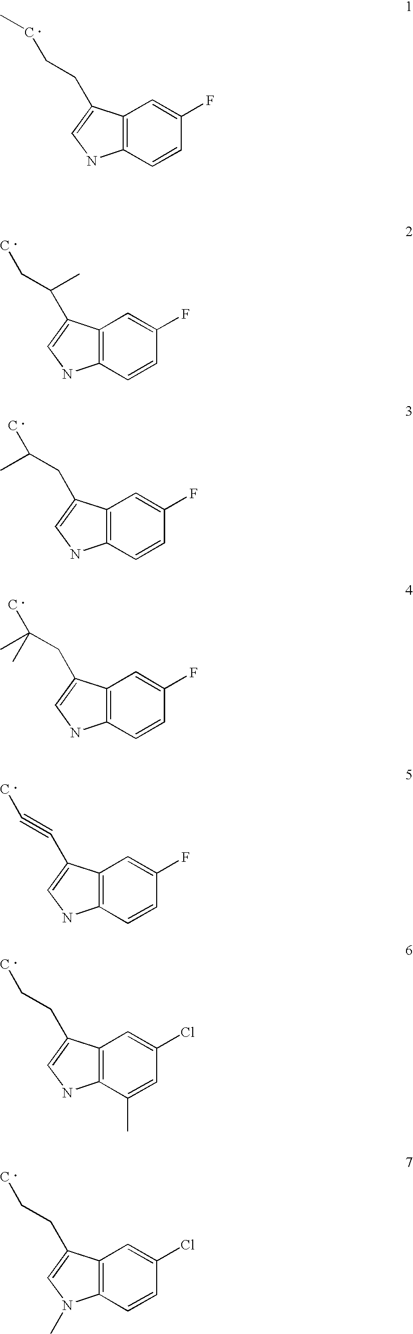

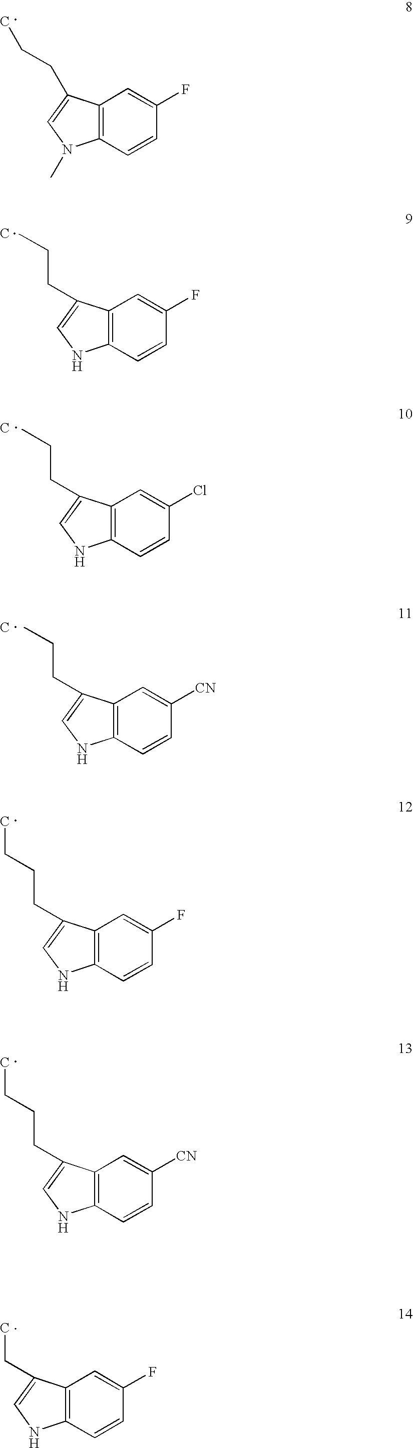

In these formulae ‘Q’, the dot represents the attachments to the phenylpiperazine part of the compounds of formula (1).
Synthesis of Q1: 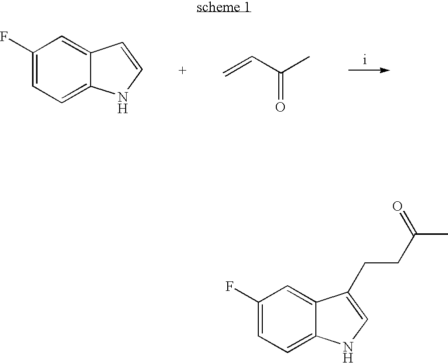
Scheme 1, Step i:

0.56 g (1.5 mmol) of CeCl₃.7H₂O and 0.22 g (1.5 mmol) of sodium iodide were together with 2.3 g of silica (SiO₂) taken up in 33 ml of acetonitrile. The resulting mixture was stirred for 14 hours. Then the mixture was concentrated in vacuo until a yellowish powder remained. Subsequently, 0.68 g (5 mmol) of 5-fluoroindole were added, then 0.35 g (5 mmol) of methylvinylketone were added, the solid mixture turned greyish after which the color returned again to yellow. After 4 hours the mixture was put on top of a flash chromatographic column (SiO₂) and eluted with DCM. 0.80 g (78%) of the indolylketone could be isolated.

The ketone was coupled to amine 1-H.HCl according to step ii in scheme B2, instead of DCE, THF was used as the solvent in the reductive alkylation.
Synthesis of Q2: 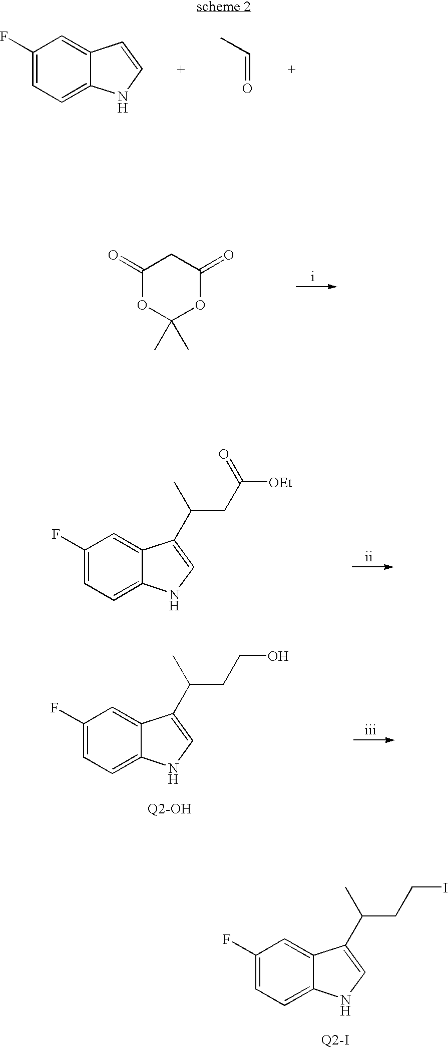
Scheme 2, Step i:

4.51 g (33.4 mmol) of 5-fluoroindole and 4.81 g (33.4 mmol) of Meldrum's acid were taken up in 40 ml of acetonitrile. Subsequently, 3.75 ml (66.8 mmol) of acetic aldehyde were added to the reaction mixture, after which stirring was continued for 24 hours. The reaction mixture was concentrated in vacuo, and dissolved again in 67 ml of pyridine after which 6.7 ml of absolute ethanol and 0.84 g of cupper powder were added. The mixture was brought to reflux for three hours. The reaction mixture was cooled and concentrated in vacuo, the residu was taken up in diethylether, the suspension filtered and the filtrate was washed with 1M HCl, 20% NH₄Cl (H₂O) and water, respectively. The organic layer was dried (MgSO₄) and concentrated in vacuo, the residu purified by flash column chromatography (SiO₂, eluent: DCM/petroleum ether 4/1), yielding 6.43 g (77%) of the indolylalkylester.

Scheme 2, Step ii:

4 g (105.3 mmol) of LiAlH₄ were taken up in 100 ml of THF, after which 8.1 g (32.5 mmol) of indolylalkylester (from step i) dissolved in 50 ml of THF, were added dropwise during 30 minutes. The reaction mixture was brougt to reflux for 45 minutes. After cooling (ice bath) a mixture of 4 ml of water in 10 ml of THF, 8 ml of 2M NaOH, and 8 ml of water were added dropwise to the reaction mixture respectively. The latter mixture was brought to reflux again for 30 minutes. After cooling the reaction mixture was filtered and the filtrate concentrated in vacuo, the residu purified by flash column chromatography (SiO₂, eluent: diethylether), yielding 6.73 g (100%) of the pure indolylalkylalcohol Q2-OH.

Scheme 2, Step iii:

To a solution of 10.64 g (40.6 mmol) of triphenylphosphine and 2.76 g (40.6 mmol) of imidazole in 500 ml of CH₂Cl₂ was added 10.31 g (40.6 mmol) of iodine and the mixture was stirred for half an hour. Subsequently a solution of 10.31 g (40.6 mmol) of the alcohol in CH₂Cl₂ was added dropwise in half an hour and stirring was continued for one hour. The reaction mixture was washed with water, 5% Na₂S₂O₃ and water after which the organic fraction was dried (Na₂SO₄). After removal of the drying agent by filtration and solvent by concentration in vacuo, the residu was subjected to flash chromatography (SiO₂, eluent: CH₂Cl₂) eventually yielding 9.83 g (76%) of the desired Q2-I.
Synthesis of Q3: 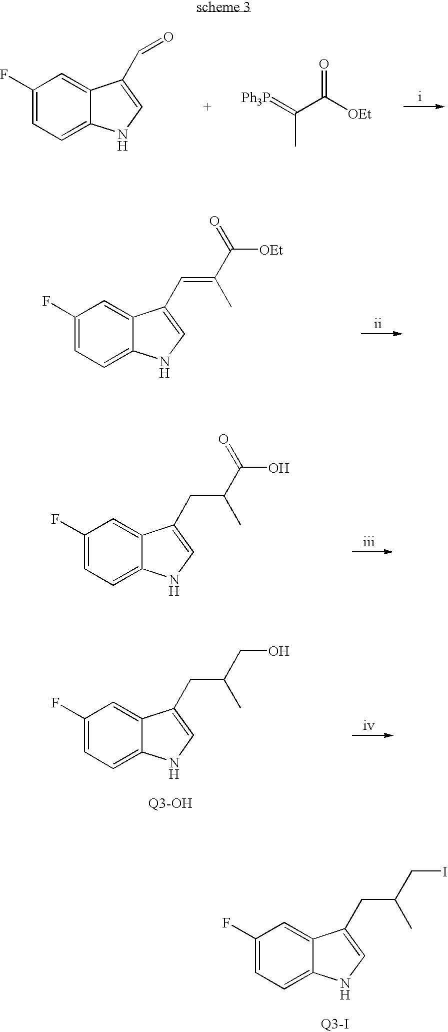
Scheme 3, Step i:

5.5 g (33.7 mmol) of 5-fluoro-3-carbaldehyde and 18.3 g (50.6 mmol) of the triphenylphosphine derivative were taken up in 165 ml of dioxane, after which the mixture was brought to reflux for 3 hours. After cooling, the reaction mixture was concentrated in vacuo and the residu purified by flash column chromatography (SiO₂, eluent: DCM), yielding 8.72 g (100%) of the pure indolylalkenylester.

Scheme 3, Step ii:

7.49 g (30.3 mmol) of the (from step i) were dissolved in 200 ml of absolute ethanol and 0.75 g of 10% Pd/C were added after which hydrogenation was started at room temperature and 1 atmosphere. After 14 hours the mixture was filtered over hyflo, the filtrate concentrated in vacuo, yielding 7.54 g (100%) of the corresponding indolylalkylester.

Scheme 3, Step iii:

3.7 g (98.2 mmol) LiAlH₄ was taken up in 100 ml of dry THF after which a solution 7.54 g (30.3 mmol) of the indolylalkylester (of step iii) in 50 ml of dry THF was added dropwise to the reaction mixture in 30 minutes. After cooling (ice bath) a mixture of 3.7 ml of water in 10 ml of THF, 7.4 ml of 2M NaOH, and 7.4 ml of water were added dropwise to the reaction mixture respectively. The latter mixture was brought to reflux again for 30 minutes: After cooling the reaction mixture was filtered and the filtrate concentrated in vacuo, the residu purified by flash column chromatography (SiO₂, eluent: diethylether), yielding 6.27 g (100%) of the pure indolylalkylalcohol Q3-OH.

Scheme 3, Step iv:

The conversion of the resulting alcohols to the corresponding iodo derivatives was performed according to the procedure described in scheme 2 step iii.
Synthesis of Q4: 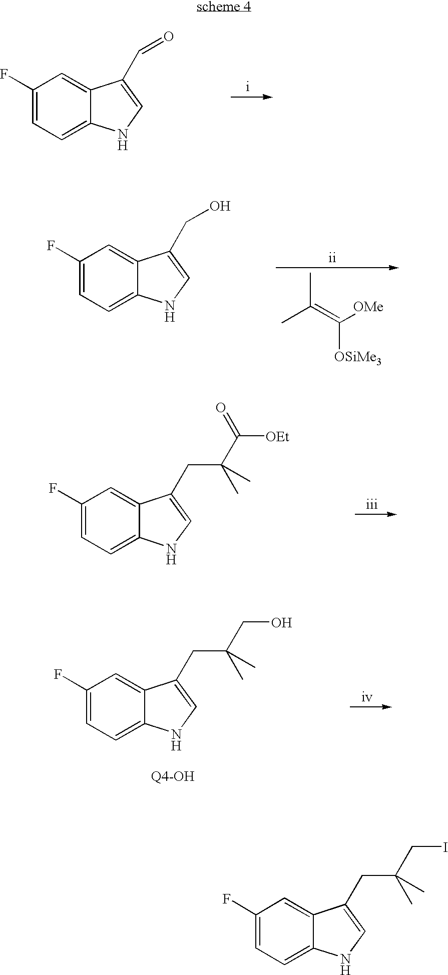
Scheme 4, Step i:

4.89 g (30 mmol) of 5-fluoro-3-carbaldehyde were solved in 100 ml of methanol and the solution cooled in an ice bath. 3.42 g (90 mmol) of NaBH₄ were added portionwise in 15 minutes. After 30 minutes the ice bath was removed after which the reaction was stirred for another 30 minutes. 400 ml of water were added after which extraction with DCM took place (4×), the collected organic fractions were filtered over a water repellant filter, the dry filtrate carefully concentrated in vacuo (T<25° C.), eventually yielding 4.95 g (100%) of the corresponding indolylmethylalcohol, which was directly used in the next step.

Scheme 4, step ii:

4.95 g (30 mmol) of the indolylmethylalcohol (from step i) were dissolved in 300 ml of DCM after which 12.2. ml (60 mmol) of 1,1-dimethyl-2methoxy-2-trimethylsilyloxyethene, and 1.76 g (3 mmol) of Mg(NTf₂)₂ hydrate were added. The mixture was stirred for one hour. Subsequently the reaction mixture was washed with water, and the organic layer was filtered over a water repellant filter, the dry filtrate carefully concentrated in vacuo. The residu was purified by flash column chromatography (SiO₂, eluent: DCM), yielding 6.9 g (92%) of the corresponding pure indolylalkyl ester.

Scheme 4, Step iii:

This step was done analogous to Scheme 3, step iii.

Scheme 4, Step iv:

The conversion of the resulting alcohols to the corresponding iodo derivatives was performed according to the procedure described in scheme 2 step iii. The compound isolated was not the iodide, but the corresponding triphenylphosphonium iodide salt which can be tranformed into the desired iodide Q4-I by refluxing the salt in butyronitrile. After work up the the crude product was purified by flash column chromatography (SiO₂, eluent: DCM).
Synthesis of Q5: 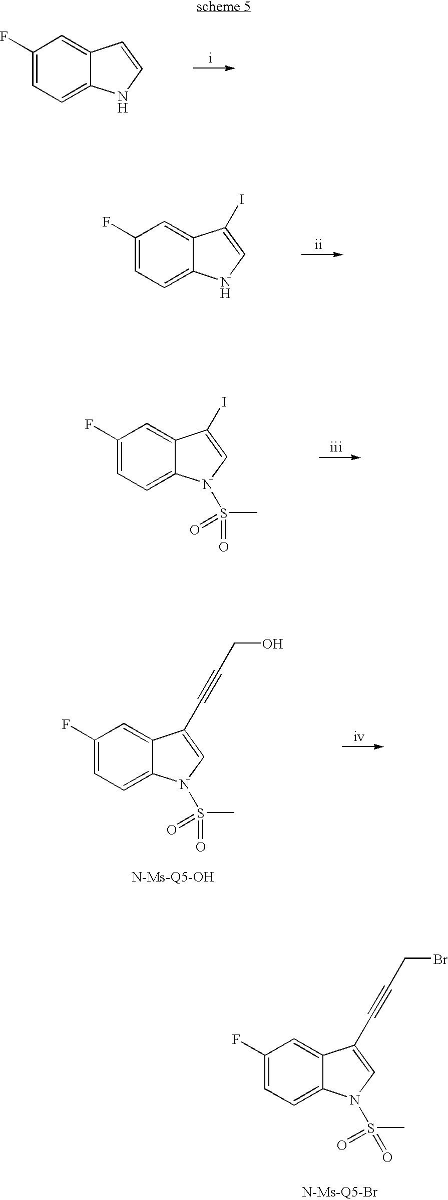
Scheme 5, Step i:

4.73 g (84.4 (mmol) of KOH were added to a cooled (water bath) solution of 3.0 g (22.2 mmol) of 5-fluoroindole in 11 ml of DMF. After 5 minutes, a solution of 5.63 g (22.2 mmol) iodine in 11 ml of DMF was added dropwise. After the addition was complete, stirring was continued for 15 minutes.

Subsequently the reaction mixture was poured into a solution containing 2.22 g of NaHSO₃, 22 ml of 25% NH₄OH and 333 ml of water. Crystallization started, filtration yielded 5.87 g of the unstable 3-indolyl-iodide, which was directly used in step ii.

Scheme 5, Step ii:

The 3-indolyl-iodide was dissolved in 33 ml of toluene, and in the following order were added: 33 ml of water, 22 ml 50% of NaOH and 0.71 g (2.22 mmol) of TBAB. While vigorously stirring, a solution of 2.8 g (24.4 mmol) of mesylchloride in 33 ml of toluene, was added. After the addition was complete, stirring was continued for 90 minutes. The reaction mixture was washed with water (2×), and the organic fraction was concentrated in vacuo, yielding 6.67 g of a light brown oil. This residu was purified by flash column chromatography (SiO₂, eluent: DCM/petroleum ether 2/3), yielding 3.92 g (almost white) of the corresponding pure N-mesyl-derivative.

Scheme 5, Step iii:

0.65 g (2 mmol) of the N-mesyl-derivative (from step ii), 0.13 g (2.4 mmol) of propargylalcohol, 55 mg (0.078 mmol) of (PPh₃)₂PdCl₂, 27 mg (0.141 mmol) of CuI, were taken up in 10 ml of triethylamine (degassed for 30 minutes). This mixture was stirred for 5 hours under an nitrogen atmosphere. Subsequently, water and diethylether were added, the water fraction was extracted with diethylether. The combined organic fractions were washed with brine, and filtered over a water repellant filter, the dry filtrate concentrated in vacuo. The residu was purified by flash column chromatography (SiO₂, eluent: DCM/MeOH 97/3), yielding 0.40 g (76%) of the pure N-Ms-Q5-OH.

Scheme 5, Step iv:

0.40 g (1.52 mmol) of 05-OH (from step iii), 480 mg (1.82 mmol) of PPh₃ and 600 mg (1.82 mmol) of tetrabromomethane, were taken up in 10 ml of DCM. The reaction mixture was stirred for 28 hours after which the reaction mixture was concentrated in vacuo and the residu purified by flash column chromatography (SiO₂, eluent: ethylacetate/petroleum ether ¼), yielding 430 mg (86%) of a light yellow oil (solidifies on standing) containing N-Ms-Q5-Br.

This bromide was used in the synthesis of compound 5. The mesylgroup of N-mesyl-compound 5 can be removed by standard procedures like refluxing (4 hours) in 1M TBAF in THF. Usual work up and purification by column chromatography yields pure compound 5.
Synthesis of Q6-Q10:

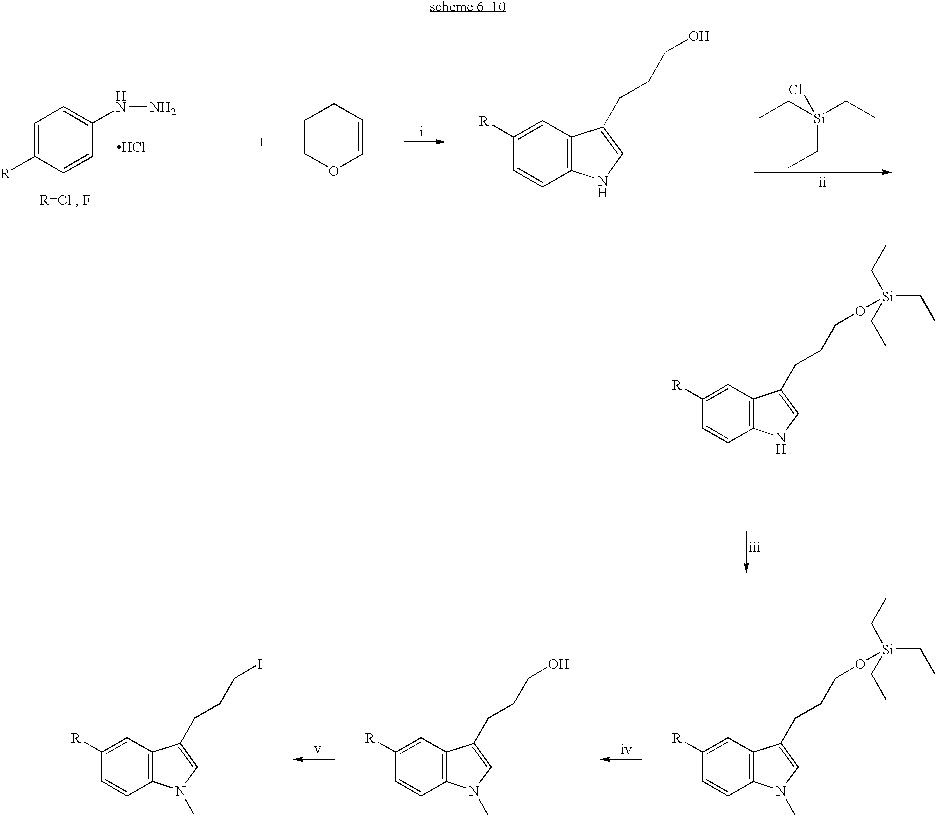

All starting hydrazines were commercially available.

Scheme 6-10, Step i:

R═Cl

A stirred suspension of 4-Chlorophenylhydrazine monohydrochloride (25 g, 139 mmol) in 260 ml of 1,2-propanediol was heated on an oil bath of 110° C. 3,4-dihydropyrane (12.5 ml, 136 mmol) was added dropwise for 15 minutes. The reaction mixture was stirred for 4.5 hours at 95-100° C. After cooling to room temperature, 150 ml of 25% NaOH were added and stirring was continued for 10 minutes. 250 ml of MTBE were added and after additional stirring for 10 min., the MTBE-layer was separated and the aqueous layer was extracted 2× with MTBE. The combined organic layers were washed with H₂O, 5% NaHCO₃ and brine respectively. The organic layer was dried (Na₂SO₄). The drying agent was removed by filtration and the solvent by evaporation under reduced pressure. The residue was chromatographed (SiO₂) using EtOAc/petroleum ether 4/1 as the eluent to give 25.6 g (87%) of the indole as a brown oil, containing Q10-OH.

Scheme 6-10, Step ii:

To a stirred solution of the indole Q10-OH of step i (25.9 g, 123 mmol) and imidazole (8.71 g, 128 mmol) in 150 ml DMF at 0 ²C was added triethylsilylchloride (21.5 ml, 128 mmol). The reaction mixture was stirred for 3 hours at room temperature H₂O and Et₂O were added. The Et₂O layer was separated and the aqueous layer was extracted 1× with Et₂O. The combined Et₂O layers were washed with H₂O (3×) and brine respectively. The Et₂O was dried (Na₂SO₄) and evaporated under reduced pressure to give 36.04 g (90%) of the silylated alcohol as a brown oil.

Scheme 6-10, Step iii:

To a stirred suspension of NaH (60%) (5.12 g, 128 mmol) in 100 ml of dry DMF a solution of the silylated alcohol of step ii (36.04 g, 107 mmol) in 50 ml dry DMF was added dropwise. Stirring was continued at room temperature for 1 h. The reaction mixture was cooled to 0° C. and a solution of MeI (8.65 ml, 139 mmol) in 50 ml dry DMF was slowly added dropwise. After addition the reaction mixture was stirred at room temperature for 18 h. H₂O was added and the aqueous layer was extracted 3× with Et₂O. The combined Et₂O layers were washed with H₂O (3×) and brine (1×) respectively. The Et₂O was dried (Na₂SO₄) and evaporated under reduced pressure. The residue was chromatographed using CH₂Cl₂/PA 1:1 as eluent to give 31.51 g (87%) of the methylated indole as a thick liquid.

Scheme 6-10, Step iv:

A mixture of the methylated indole (31.5 g, 90 mmol) and 1.0M (in THF) TBAF (117 ml, 117 mmol) was stirred at room temperature for 20 h. H₂O and Et₂O were added. The Et₂O layer was separated and the aqueous layer was extracted 1× with Et₂O. The combined Et₂O layers were washed with H₂O (3×) and brine respectively. The Et₂O was dried (Na₂SO₄) and evaporated under reduced pressure. 200 ml petroleum ether was added to the residue and the suspension was filtered off by suction to give 17.19 g (85%) as an off white solid, containing Q7-OH.

Scheme 6-10, Step v:

The conversion of the resulting alcohols to the corresponding iodo derivatives was performed analogously to the procedure described in scheme 2 step iii.

Q6-OH, Q8-OH and Q9-OH can be synthesized analogously to the previous procedures.
Synthesis of Q11:

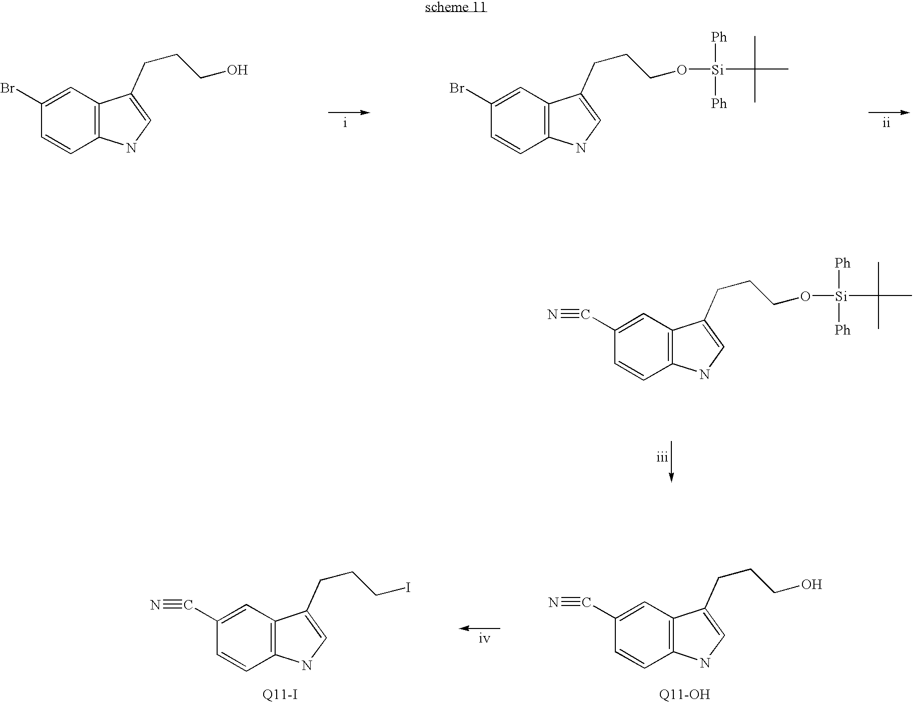

The starting 5-bromoindole alcohol was prepared according to: Campos, Kevin R.; Woo, Jacqueline C. S.; Lee, Sandra; Tillyer, Richard D., Org. Lett., 6 (2004) 79-82.

Scheme 11, Step i:

A solution of 45 g of the indolylpropylalcohol (0.177 mol) and 12.65 g of imidazole (0.185 mol) in 150 ml of DMF was cooled in an ice/EtOH bath and tert.-butyldiphenylsilylchloride (50.8 g, 48.1 ml, 0.185 mol) was added in two portions. The reaction mixture was stirred at 0° C. for one hour after which it was allowed to reach room temperature. After 4 hours stirring at room temperature, the reaction mixture was poured into water after which the resulting mixture was extracted two times with Et₂O. The combined extracts were washed with water (3 times), brine, and dried on Na₂SO₄. After removal of the drying agent by filtration and solvent by concentration in vacuo, the residu was subjected to flash column chromatography (SiO₂, eluent: DCM/PA=1/1) giving the corresponding silylated alcohol (70.9 g, 0.144 mol) as a light orange viscous oil.

Scheme 11 Step ii:

A mixture under a nitrogen atmosphere of the silylated alcohol (42.4 g, 86.2 mmol), CuI (1.64 g, 8.6 mmol), Palladium tetrakis (5 g, 4.31 mmol) and potassium cyanide (11.17 g, 172.4 mmol) in 110 ml butyronitrile was refluxed for 6 h. The reaction mixture was cooled to room temperature and filtered through a pad of Hyflo. After rinsing the pad of Hyflo with 750 ml EtOAc, the organic layer was washed with H₂O (2×) and brine (1×). The organic layer was evaporated under reduced pressure and the residue was chromatographed (SiO₂) with CH₂Cl₂/petroleum ether 3/1 as eluent to give 35.6 g (94%) of the cyanated indole as a light yellow solid.

Scheme 11 Step iii:

A mixture of the cyanated indole (35.6 g, 81.1 mmol) and 105.3 ml 1.0 M TBAF (in THF) was stirred at room temperature for 20 h. The solvent was evaporated under reduced pressure and 750 ml CH₂Cl₂ was added to the residue. The CH₂Cl₂ fraction was washed with H₂O (3×). The product started to crystallize from the organic layer. The CH₂Cl₂ fraction was separated and stirred in an ice/EtOH bath for 30 minutes. The resulting suspension was filtered by suction to give 13.7 g (72%) of the alcohol as nearly white solid.

Scheme 11 Step iv:

Was prepared according to the procedure as described for scheme 2 step iii.
Synthesis of Q12:

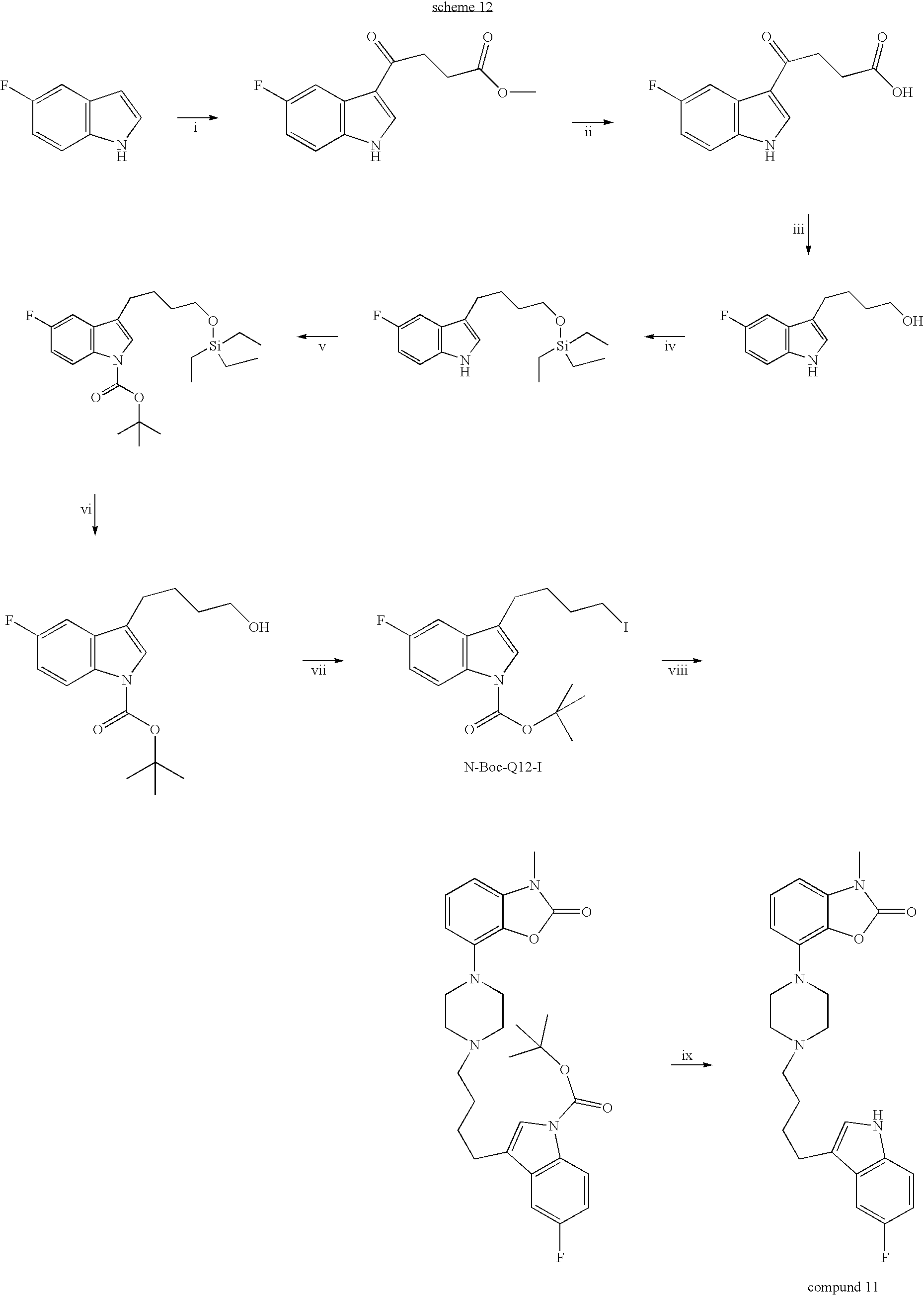

The 5-fluoroindole was commercially available.

Scheme 12 Step i:

To a cooled solution of 5-fluoroindole (3 g, 22.2 mmol) in 100 ml CH₂Cl₂ at 0° C. was added 33.8 ml 1.0 M Et₂AlCl in hexane. The resulting light yellow solution was stirred at the same temperature for 30 minutes after which a solution was added of 3-carbomethoxypropionylchloride (4.1 ml, 33.3 mmol) in 50 ml CH₂Cl₂ at 0° C. After the complete addition the color of the solution was changed to orange and stirring was continued for another 2.5 hours at the same temperature. The reaction mixture was poured into 500 ml of Hamilton pH 7 buffer (gas evolution) and the aqueous layer was extracted with 750 ml (in total) CH₂Cl₂ (3×). The combined organic layers were washed with H₂O (1×) and brine (1×). The CH₂Cl₂ fraction was dried (Na₂SO₄). The drying agent was removed by filtration and the solvent by evaporation under reduced pressure. The residue was chromatographed (SiO₂) with EtOAc/PA 1/1 as eluent to give 3.82 g (69%) of the acylated indole as a light colored solid.

Scheme 12 Step ii:

A mixture of the acylated indole (3.46 g, 13.9 mmol) and 28 ml 1.0 N NaOH in 75 ml THF/MeOH 2/1 was stirred at room temperature for 2.5 h. The mixture was acidified under cooling with 25 ml 1.0 N HCl. The resulting suspension was filtered by suction to give 2.42 g (74%) of the acid as an off-white solid.

Scheme 12 Step iii:

Was prepared according to the procedure as described for scheme 14 step ii.

Scheme 12 Step iv:

Was prepared according to the procedure as described for scheme (18, 51-52, 94-95) step ii. Additionally the residue was chromatographed (SiO₂) with CH₂Cl₂ as the eluent.

Scheme 12 Step v:

To a stirring solution of the silylated alcohol (3.43 g, 10.7 mmol) in 30 ml CH₃CN was added Boc₂O (3.50 g, 16 mmol) and DMAP (0.13 g, 1.07 mmol). The yellow solution was stirred at room temperature for 30 minutes after which imidazole (0.98 g, 16 mmol) was added. Stirring was continued at room temperature for 1.5 hours after which 55 ml CH₂Cl₂ was added. The CH₂Cl₂ fraction was washed with 0.5% HCl (3×) and dried (MgSO₄). The drying agent was removed by filtration and the solvent by evaporation under reduced pressure to give 4.09 g (91%) of the carbamated indole as a thick yellow liquid.

Scheme 12 Step vi:

A mixture of the carbamated indole (4.09 g, 9.7 mmol) and 1.0 M (in THF) TBAF (12.6 ml, 12.6 mmol) was stirred at room temperature for 4 hours. H₂O and Et₂O were added. The Et₂O layer was separated and the aqueous layer was extracted with Et₂O (2×). The combined Et₂O layers were washed with H₂O (2×) and brine (1×). The Et₂O fraction was dried (by a Water Reppelling Filter) and concentrated in vacuo under reduced pressure. The residue was chromatographed (SiO₂) with CH₂Cl₂/MeOH 99:1 as eluent to give 4.04 g (87%) as a yellow oil.

Scheme 12 Step vii:

Was prepared according to the procedure as described for scheme 2 step iii.

Scheme 12 Step viii:

Was prepared according to the procedure as described for scheme A2 step i.

Scheme 12 Step ix:

A mixture of the carbamated indole (3.15 g, 6 mmol), anisole (0.65 ml, 6 mmol) and 60 ml 1.0 M AcCl/EtOH stirred at 60° C. for 20 h. The reaction mixture was cooled to room temperature and the suspension was filtered by suction and the filter was washed with EtOH to give 2.18 g (79%) of the indole as an off-white solid containing compound 11. Melting point: 254-256° C.

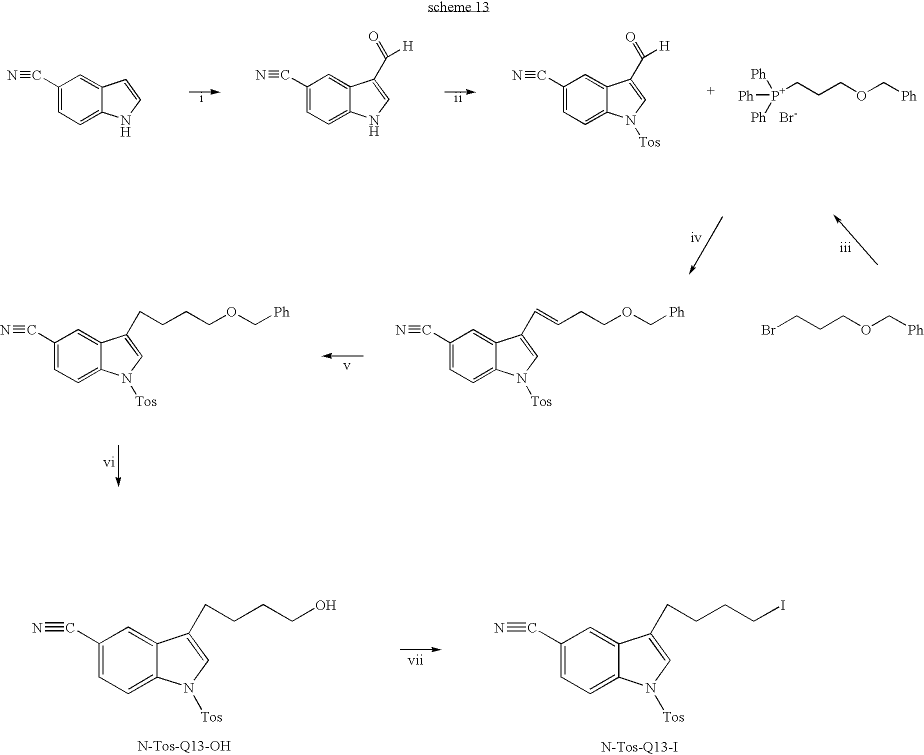

The starting material was commercially available.

Scheme 13 Step i:

POCl₃ was added dropwise to 200 ml dry DMF at 15° C. The resulting dark pink solution was stirred for 20 min after which it was cooled to 0-5° C. To this solution was added dropwise a solution of 5-cyanoindole (20 g, 140 mmol) in 45 ml dry DMF. After 10 min of stirring a very thick suspension was formed. The reaction mixture was allowed to warm to room temperature and stirred for 4 h. Next the reaction mixture was poured into a 650 ml saturated Na₂CO₃/ice mixture. The resulting suspension was stirred for 30 minutes after which it was filtered by suction and dried (by use of an oven) to give 29.8 g yellow solid. To this solid was added 80 ml EtOAc and the suspension was filtered again by suction to give 21.79 g (79%) of the formylated indole as a solid.

Scheme 13 Step ii:

A mixture of the formylated indole (1.7 g, 10.3 mmol), tosylchloride (2.99 g, 15.7 mmol) and Et₃N (25 ml, 17.99 mmol) was refluxed for 1.5 h. The resulting very thick suspension was cooled to room temperature and 35 ml ice water was added. The reaction mixture was left to stand at 4° C. for 1 hour after which the suspension was filtered by suction. The solid was further purified by recrystallisation from EtOAc to give 1.58 g of tosylated product.

The filtrate was evaporated under reduced pressure and the residue was chromatographed with CH₂Cl₂/PA 4:1→CH₂Cl₂ as eluent to give 0.5 g of the tosylated product. This sample was identical and added to the previously isolatedsolid to give in total 2.08 g (64%) of the tosylated product as a white solid.

Scheme 13 Step iii:

A mixture of the bromide (40 g, 174 mmol) and PPh₃ (45.7 g, 174 mmol) was refluxed in 200 ml toluene for 16 h. After cooling to room temperature the resulting slurry (which could not be filtered), was warmed to reflux again and cooled to room temperature while the mixture was vigorously stirred. A very hard white solid crystallized from the solution. It was pulverized and filtered by suction. The solid was recrystallized from CH₃CN/petroleum ether to give 58.1 g (68%) of the corresponding phosphonium salt.

Scheme 13 Step iv:

To a stirring suspension of the phosphonium salt (41.77 g, 85 mmol) in 500 ml dry THF at −10° C. was added 85 ml 1.0M (in THF) NaHMDS in 45 min. After complete addition the reaction mixture was stirred for 1.5 hours at the same temperature. The reaction mixture was further cooled to −65° C. and the tosylated formylindole (27.5 g, 84.7 mmol) was added in portions in 75 minutes by means of an addition funnel for solids. After the complete addition the reaction mixture was allowed to warm to room temperature and stirred for 22 h. 1 L of ice water and 500 ml of Et₂O were added to the reaction mixture and after separation the aqueous layer was extracted with Et₂O (2×). The combined organic layers were washed with 500 ml H₂O (1×) and 400 ml brine (1×). The Et₂O fraction was evaporated under reduced pressure and the residue was chromatographed (SiO₂) with CH₂Cl₂ as the eluent to give 16.89 g (44%) of the alkene as an off-white solid.

Scheme 13 Step v:

A mixture of the alkene (11 g, 23 mmol) and 0.5 g 10% Pd/C in 240 ml EtOAc/MeOH 1/1 was hydrogenated (1 atm) at room temperature for 4 h. The reaction mixture was filtered through a pad of Hyflo which was rinsed with 200 ml EtOAc/MeOH 3/1. The filtrate was evaporated under reduced pressure to give 11.2 g (103%) as a solid.

Scheme 13 Step vi:

To a cooled clear orange solution of the tosylated indole (11.2 g, 23 mmol) in 150 ml CH₂Cl₂ at −75° C. was added dropwise 100 ml 1.0M BCl₃ (in CH₂Cl₂) in 1 hour during, which the temperature was kept below −60° C. Stirring was continued at −75° C. for 2 h. The resulting pink suspension was allowed to warm to room temperature and stirred for 20 h. The reaction mixture was cooled in an ice bath and 550 ml 5% NaHCO₃ was carefully added during which the temperature was kept below 20 L° C. and the pH rose until 8. The aqueous layer was extracted with 300 ml CH₂Cl₂ (2×). The combined organic layers were washed with H₂O (1×) and brine (1×) and dried (Na₂SO₄). The drying agent was removed by filtration and the solvent by evaporation under reduced pressure. The residue was chromatographed (SiO₂) with CH₂Cl₂/MeOH 98/2 as eluent to give 7.39 g (87%) of the de-benzylated alcohol as a solid.

Scheme 13 Step vii:

Was prepared analogously to step iii in scheme 2. The obtained iodide can be coupled to an amine following the procedure in schema A2, step i. The resulting N-tosylated product can be de-tosylated by standard procedures like refluxing (72 hours) in 1M TBAF in THF. Usual work up and purification by column chromatography yield the pure product like compound 12.
Synthesis of Q14: 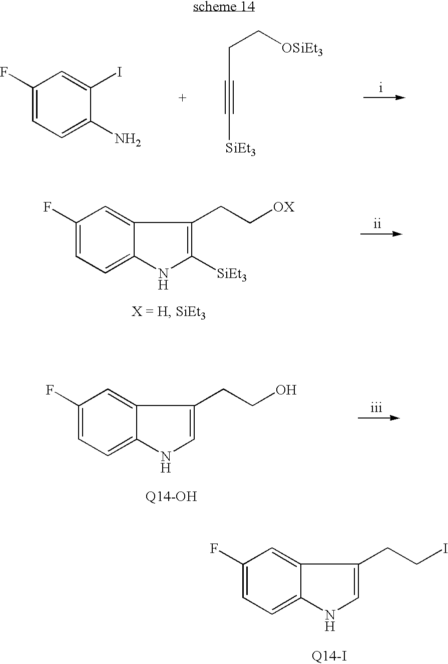
Scheme 14, Step i:

2-iodo-4-fluoroaniline (2.70 g, 11.4 mmol), 4-triethylsilyl-1-(triethylsilyloxy)-3-butyne (3.82 g, 12.5 mmol), LiCl (0.48 g, 11.4 mmol), Na₂CO₃ (2.18 g, 20.5 mmol), Pd(OAc)₂ (0.128 g, 0.57 mmol) were suspended in 120 ml DMF and nitrogen was bubbled through the suspension for 45 minutes. The mixture was heated to 100° C. in an oilbath and stirred at this temperature for 16 hours after which it was allowed to reach room temperature and subsequently concentrated in vacuo. The residu was taken up in some dichloromethane and filtered over Celite. Flash chromatography on silica (eluent: diethyl ether/petroleum ether 1/3) afforded a mixture of unprotected and triethylsilyl protected 2-triethylsilyl-5-fluoro-tryptophol (2.09 g, 6.02 mmol).

Scheme 14, Step ii:

A mixture of unprotected and triethylsilyl protected 2-triethylsilyl-5-fluoro-tryphtol (2.74 g, 7.83 mmol) and 15.7 ml of a 1.0 N solution of TBAF in THF were stirred for 48 hours at room temperature. Diethyl ether and water were added and the fractions were separated. The water layer was extracted twice with diethyl ether. The combined organic extracts were washed with water, brine and dried (Na₂SO₄). After removal of the drying agent by filtration and solvent by concentration in vacuo, the residu was subjected to flash chromatography (SiO₂, eluent: DCM/MeOH 97/3) affording Q14-OH, 5-fluoro-tryptophol (1.14 g, 6.36 mmol).

Scheme 14, Step iii:

The conversion of the alcohol Q14-OH to the corresponding iodo-derivate was performed according to the synthesis given in scheme 2 step iii, yielding Q14-I.

The specific compounds of which the synthesis is described above are intended to further illustrate the invention in more detail, and therefore are not deemed to restrict the scope of the invention in any way. Other embodiments of the invention will be apparent to those skilled in the art from consideration of the specification and practice of the invention disclosed herein. It is thus intended that the specification and examples be considered as exemplary only, with a true scope and spirit of the invention being indicated by the claims.

ABBREVIATIONS

| AcCl | acetylchloride | | |
|---|---|---|---|
| ADDP | 1,1′-(azodicarbonyl)dipiperidine | | |
| CDI | carbonyldiimidazol | | |
| Dba | see Huang et al., J. Am.Chem.Soc., 125(2003)6653 | | |
| DCE | dichloroethane | | |
| DCM | dichloromethane | | |
| DIAD | diisopropyldiazodicarboxylate | | |
| DIPE | diisopropylether | | |
| DIPEA | diisopropylethylamine | | |
| | CH₂Cl₂(ml) | MeOH(ml) | NH₄OH(ml) |
| DMA 0.125 | 980 | 18.75 | 1.25 |
| DMA 0.187 | 970 | 28.13 | 1.87 |
| DMA 0.25 | 960 | 37.5 | 2.5 |
| DMA 0.50 | 920 | 75.0 | 5.0 |
| DMA 0.75 | 880 | 112.5 | 7.5 |
| DMA 1.00 | 840 | 150.0 | 10.0 |

| | |
|---|---|
| DMAP | -dimethylaminopyridin |
| DME | dimethoxyethane |
| DMF | N,N-dimethylformamide |
| EtOH | ethanol |
| MeOH | methanol |
| MTBE | methyl(tert.)-butylether |
| NMP | N-methylpyrrolidon |
| PA | petroleum ether |
| TBAB | tetrabutylammoniumbromide |
| TBAC | tetrabutylammoniumchloride |
| TBAF | tetrabutylammoniumfluoride |
| THF | tetrahydrofurane |
| XPHOS | see Huang et al., J. Am.Chem.Soc., 125(2003)6653 |

Example: Formulation of Compound 4 Used in Animal Studies

For oral (p.o.) administration: to the desired quantity (0.5-5 mg) of the solid compound-4 in a glass tube, some glass beads were added and the solid was milled by vortexing for 2 minutes. After addition of 1 ml of a solution of 1% methylcellulose in water and 2% (v/v) of Poloxamer 188 (Lutrol F68), the compound was suspended by vortexing for 10 minutes. The pH was adjusted to 7 with a few drops of aqueous NaOH (0.1N). Remaining particles in the suspension were further suspended by using an ultrasonic bath.

For intraperitoneal (i.p.) administration: to the desired quantity (0.5-15 mg) of the solid compound 4 in a glass tube, some glass beads were added and the solid was milled by vortexing for 2 minutes. After addition of 1 ml of a solution of 1% methylcellulose and 5% mannitol in water, the compound was suspended by vortexing for 10 minutes. Finally the pH was adjusted to 7.

Example: Pharmacological Testresults

TABLE 2
In vitro affinities and functional activity of compounds of the invention
Dopamine-D₂ and serotonin reuptake receptor affinity data obtained
according to the protocols given above are shown in the table below.
In vitro functional activity at cloned human dopamine D_{2,L} receptors
as measured by accumulation of radiolabeled cAMP (potency: pEC₅₀,
intrinsic activity ε)

| | Dopamine-D₂ | 5-HT reuptake | Dopamine-D₂ |
|---|---|---|---|
| | binding | binding | cAMP accum |
| No | pKᵢ | pKᵢ | ε (intrinsic activity) |
| 1 | 7.7 | 8.3 | 0.13 |
| 2 | 6.8 | 9.0 | |
| 3 | 7.0 | 9.1 | 0.43 |
| 4 | 8.1 | 8.6 | 0.22 |
| 6 | | 8.0 | 0.52 |
| 7 | 6.7 | 8.6 | 0.15 |
| 8 | 7.5 | <8.0 | 0.44 |
| 9 | 7.1 | 8.1 | 0.12 |
| 10 | 6.9 | 8.4 | 0.66 |
| 11 | 7.6 | <8.0 | 0.72 |
| 12 | 8.1 | >9.0 | 0.75 |
| 13 | 8.4 | 8.5 | 0.22 |
| 15 | 7.1 | 9.5 | |
| 16 | 7.4 | >9.0 | 0.15 |
| 17 | 7.8 | 8.3 | 0.32 |

1. Compounds of the general formula (1): 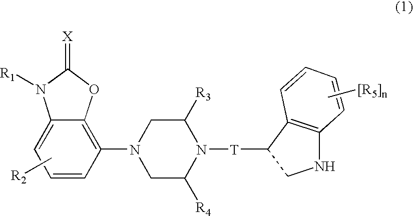
wherein:
X═S or O,
R₁ is H, (C₁-C₆)alkyl, CF₃, CH₂CF₃, OH or O—(C₁-C₆)alkyl
R₂ is H, (C₁-C₆)alkyl, halogen or cyano
R₃ is H or (C₁-C₆)alkyl
R₄ is H, (C₁-C₆)alkyl, optionally substituted with a halogen atom
T is a saturated or unsaturated carbon chain of 2-7 atoms, wherein one carbon atom may be replaced with a nitrogen atom, optionally substituted with an (C₁-C₃)-alkyl, CF₃ or CH₂CF₃ group, an oxygen atom or a sulphur atom, which chain is optionally substituted with one or more substituents selected from the group consisting of (C₁-C₃)alkyl, (C₁-C₃)alkoxy, halogen, cyano, trifluoromethyl, OCF₃, SCF₃, OCHF₂ and nitro,
the dotted line is either a single or a double bond,
R₅ is a substituent selected from the group consisting of (C₁-C₃)alkyl, (C, —C₃)alkoxy, halogen, cyano, trifluoromethyl, OCF₃, SCF₃, OCHF₂ and nitro,
n has the value 0-4,
and tautomers, stereoisomers and N-oxides thereof, as well as pharmacologically acceptable salts, hydrates and solvates of said compounds of formula (1) and its tautomers, stereoisomers and N-oxides,
with the proviso that when X═O, R₁, R₃ and R₄ are hydrogen, R₂ is hydrogen or halogen, and the group attached to T is an indolyl group, said indolyl group is substituted with one or more substituents selected from the group consisting of trifluoromethyl, OCF₃, SCF₃, OCHF₂ or nitro.

2. Compounds as claimed in claim 1, wherein the phenylpiperazine part of the compounds of formula (1) is selected from the group consisting of: 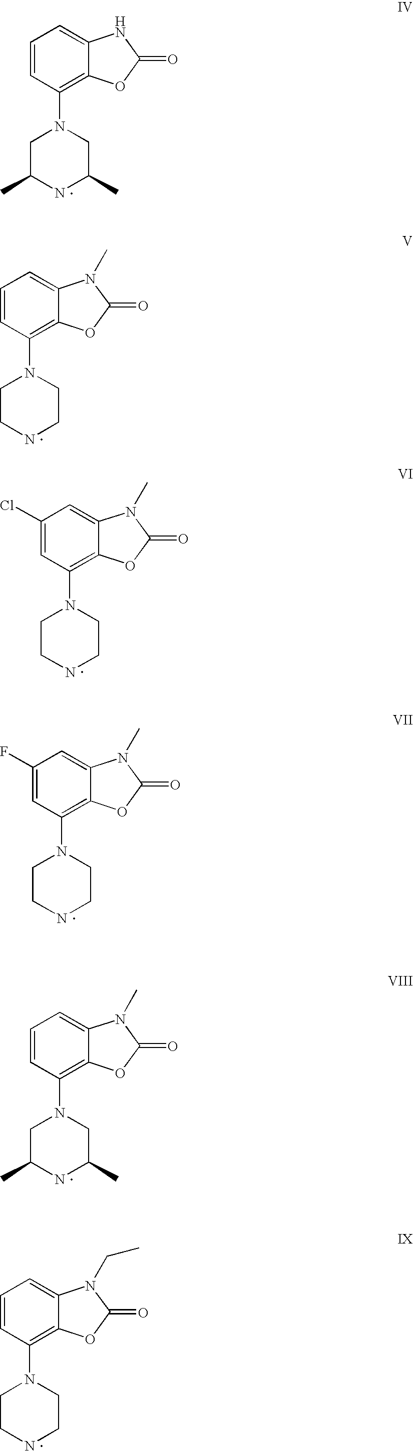 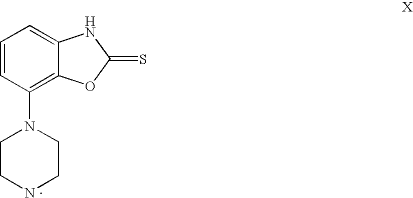
and wherein the second part of the molecule, represented by the symbols: 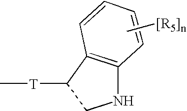
in formula (1), is selected from the group consisting of: 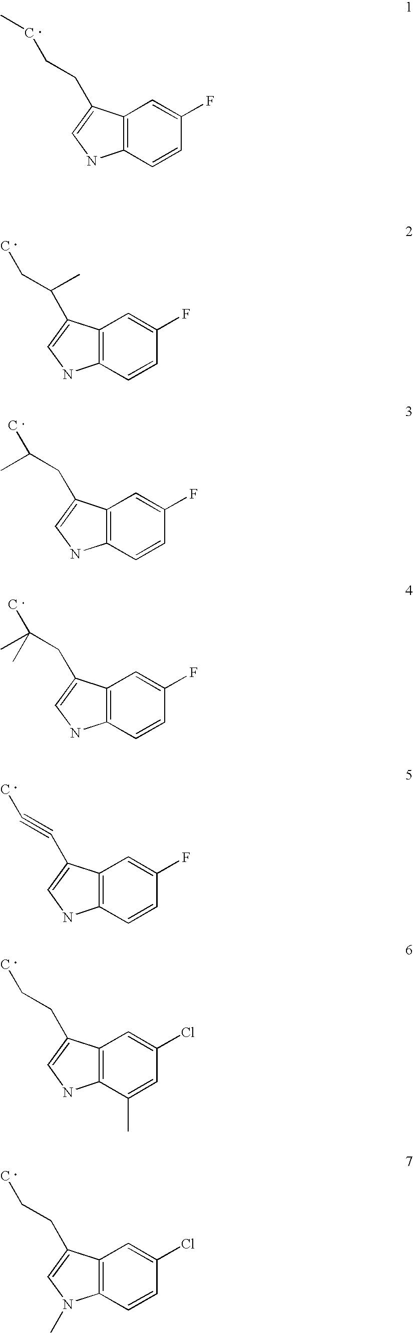 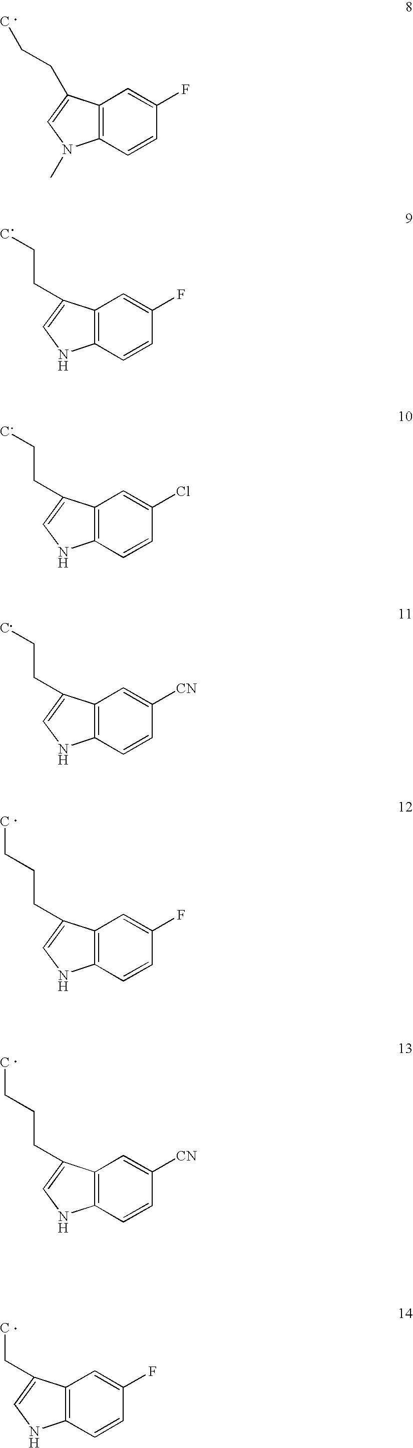
and tautomers, stereoisomers and N-oxides thereof, as well as pharmacologically acceptable salts, hydrates and solvates of said compounds of formula (1) and its tautomers, stereoisomers and N-oxides.

3. A pharmaceutical composition comprising, in addition to a pharmaceutically acceptable carrier and/or at least one pharmaceutically acceptable auxiliary substance, a pharmacologically active amount of at least one compound as claimed in claim 1, or a salt thereof, as an active ingredient.

4. A method of preparing a composition as claimed in claim 3, characterised in that at least one compound as claimed in claim 1, or a salt thereof, is brought into a form suitable for administration.

5. A compound as claimed in claim 1, or a salt thereof, for use as a medicament.

6. Use of a compound as claimed in claim 1, for the preparation of a pharmaceutical composition for the treatment of CNS disorders.

7. Use as claimed in claim 6, characterized in that said disorders are aggression, anxiety disorders, autism t, vertigo, depression, disturbances of cognition or memory, Parkinson's disease, schizophrenia and other psychotic disorders.

8. Use as claimed in claim 6, characterized in that said disorder is depression.

9. Use as claimed in claim 6, characterized in that said disorders are schizophrenia and other psychotic disorders.

10. Use as claimed in claim 6, characterized in that said disorder is Parkinson's disease.
